(19) United States
(12) Patent Application Publication
(10) Pub. No.: US 2007/0087364 A1
(43) Pub. Date: Apr. 19, 2007

(54) In vitro erythroid micronucleus assay for genotoxicity

(75) Inventors: Joe Shuga, Cambridge, RI (US); Linda Griffith, Cambridge, MA (US); Harvey F. Lodish, Brookline, MA (US); Leona D. Samson, Newton, MA (US); Dharini M. Shah, Cambridge, MA (US); Jing Zhang, Newton, MA (US)

Correspondence Address: RONALD I. EISENSTEIN;NIXON PEABODY LLP, 100 SUMMER STREET, BOSTON, MA, 02110, US

(73) Assignee: Massachusetts Institute of Technology, Cambridge, MA (US)

(21) Appl. No.: 11/527,999

(22) Filed: Sep. 27, 2006

Related U.S. Application Data
(60) Provisional application No. 60/720,812, filed on Sep. 27, 2005.

(51) Int. Cl. C12Q 1/68

(52) U.S. Cl. 435/6

(57) ABSTRACT

The present invention is directed to novel assays to measure the genotoxic effects of compounds on erythroid cells in vitro.

### CROSS REFERENCE TO RELATED APPLICATIONS

The present application claims priority under 35 U.S.C. §119(e) from U.S. Ser. No. 60/720,812, filed Sep. 27, 2005, the contents of which are incorporated herein by reference.

### FIELD OF THE INVENTION

The present invention is directed to novel assays that can measure the genotoxic effects of compounds on hematopoietic cells such as erythroid cells in vitro, particularly human erythroid cells.

### BACKGROUND OF THE INVENTION

Assays that can be used to predict toxicity are an important part of drug development, because screening candidate therapeutics for toxic and carcinogenic effects is an essential part of preclinical testing and characterization. Many prospective drugs fail in phase I clinical trials; therefore, there is a need for models that can more accurately predict human responses. This initial risk-assessment through toxicity testing both protects patients and provides early data regarding the compounds' biological effect. Prior to clinical human exposure, studies in animals and in in vitro systems are required by a variety of global regulatory agencies to assess possible health hazards (Casarett, Doull et al. 2001). However, many of these preclinical screens are costly in terms of both time and resources. Accordingly, a need exists to develop high-throughput toxicity screens early in development, before regulatory tests are conducted on a large scale to fulfill regulatory mandates. Both positive and negative results in such a prescreen can reduce wasted resources by providing early indications of problems that might ultimately lead to product failure in clinical trials.

Several different genetic toxicology assays are available. However, the traditional toxicity assays are labor intensive and/or require in vivo assays. In addition, although several of the in vitro assays are capable of detecting some genotoxins, they have problems because they are all imperfect models of human physiology. For example, the Ames assay screens mutagens using mutant Salmonella strains that are cultured under conditions such that colony formation will only occur if a mutation converts these strains from histidine-dependency back to prototrophy (Ames, Durston et al. 1973; Ames, McCann et al. 1975). Although the Ames test is well characterized, and incubation with liver microsomes allows the assay to screen promutagens that require metabolic activation, mammalian cells and bacterial cells, such as those used in the Ames test, are known to differ in their response to genetic damage (Sancar, Lindsey-Boltz et al. 2004). Some bacterial repair enzymes, such as photolyase, do not fumction at all in placental mammals, and others enzymes, such as the adaptive (ada) gene-product, share only some functions with their mammalian homologs.

Analysis of chromosome aberrations in Chinese hamster ovary(CHO) cells is one common cytogenetic technique. In this assay, CHO cells are exposed to a test agent during the DNA synthesis (S) phase of the cell cycle, and chromosome aberrations which arise during mitosis are scored (Kirkland, Gatehouse et al. 1990; Casarett, Doull et al. 2001). CHO cells have a stable, well-defined karyotype, a low number of large chromosomes, and a short cell cycle, making them well suited for visualization of chromosome aberrations. However, the in vitro chromosome aberration assay requires extensive technical expertise, and is not suitable to high throughput screening.

Another approach to assessing the genotoxic effects of a compound is the analysis of micronucleus (MN) formation. Absent in healthy cells, micronuclei are formed upon cell division in cells with DNA double-strand break(s) or dysfumctional mitotic spindle apparatus. Micronuclei are small particles consisting of acentric fragments of chromosomes or entire chromosomes, which lag behind at anaphase of cell division. After telophase, these fragments may not be included in the nuclei of daughter cells and form single or multiple micronuclei in the cytoplasm.

The cytokinesis-block micronucleus (CBMN) assay employs cytochalasin-B (Cyt-B) in vitro to interrupt cell division after telophase (Fenech and Morley 1985; Fenech 2000). Cyt-B allows cells that have undergone a cell division to be identified by their binucleate appearance, and the presence of a nuclear body excluded from the daughter nuclei clearly indicates prior DNA damage. The CBMN test is typically applied to cultured human lymphocytes or mammalian cell lines. However, CBMN test results are difficult to interpret because the test compound is always administered along with Cyt-B, which is also a toxin capable of fragmenting DNA (Kolber, Broschat et al. 1990). Finally, any test conducted in CHO cells or in other immortalized cell lines is imperfect because these cells carry mutations in genes that normally monitor genetic fidelity and regulate cell proliferation. Therefore, some compounds yield anomalous results, testing negative in all of these in vitro systems before yielding a positive response in vivo (Galloway 2004). In fact, data from the FDA indicate that approximately 30% of candidate therapeutics fail Phase I clinical trials, and approximately 75% fail in clinical trials overall.

The rodent-based micronucleus (MN) assay has become the most widely used in vivo system for evaluating the clastogenic and aneugenic potential of chemicals (Casarett, Doull et al. 2001). These rodent-based tests are most typically performed as erythrocyte-based assays. Since erythroblast precursors are a rapidly dividing cell population, and their nucleus is expelled a few hours after the last mitosis, MN-associated chromatin is particularly simple to detect in reticulocytes and normochromatic erythrocytes given appropriate staining (e.g., acridine orange). However, each MN assay requires one animal to generate one data point, i.e. to test a single test agent at a given dose. Following sacrifice of the animal, 2000 newly-synthesized erythrocytes, which are normally enucleated, are typically examined for the presence of micronuclei (MNs), which, when present, indicate genetic damage during erythropoiesis. However, the total population of these cells in the animal is larger than 2000 by several orders of magnitude. For example, approximately 100×10⁹ erythrocytes are produced each day in adult humans. Thus, the in vivo rodent erythrocyte MN assay cannot be used to test the effects of test compounds on human tissue, and is highly inefficient, requiring several animals to adequately test a given test compound, and not maximizing use of the erythrocytes in each animal.

Accordingly, there is a need for an improved assay with increased efficiency which can measure the genotoxic effects of compounds on human tissue.

### SUMMARY OF THE INVENTION

We have now discovered an assay to measure the genotoxic effects of compounds on differentiating hematopoietic cells such as erythroid progenitor cells in vitro.

Accordingly, the present invention provides novel methods to determine the genotoxic effect of a test compound on hematopoietic cell. The method involves culturing a starting population which contains an undifferentiated hematopoietic cell such as erythroid progenitor cells in vitro, for a sufficient time and under sufficient conditions to obtain erythropoietic growth. A progenitor cell can be induced to undergo erythropoiesis. A test compound is added to the culture medium. The cells are harvested and the presence of micronuclei (MN) in the cells is determined. Higher levels of MN in cells exposed to a test compound, relative to a control population of cells not exposed to the test compound, indicates the genotoxic effect of said test compound. In one embodiment, analysis of total or erythroid-specific cell numbers indicates the cytotoxic effect of the test compound.

The erythroid progenitor cells used in the methods of the invention are cells that can be induced to undergo erythropoiesis in vitro, including cells near the colony-forming unit erythroid (CFU-E) stage of erythropoiesis, the burst-forming unit erythroid (BFU-E) stage of erythropoiesis, the CFU-granulocyte erythroid macrophage megakarocyte (CFU-GEMM) stage of erythropoiesis, the long-term repopulating hematopoietic stem cell (LT-HSC) and combinations thereof. The starting population of cells is substantially free of mature cells such as mature granulocytes, reticulocytes, macrophages, T cells, B cells, and erythrocytes. Preferably, the starting population is substantially free of all cells expressing at least one of the Lin group of cell surface markers selected from the group consisting of Gr-1, Mac-1, CD3, B-220, and Ter-119 are also referred to as the lineage (Lin) group of cell surface markers. These surface markers are characteristic of differentiated hematopoietic lineages including Gr-1 (granulocytes), Ter-119 (reticulocytes and erythrocytes), Mac-1 (macrophages), CD3 (T cells), and B220 (B cells). As used herein substantially free means that 15% or less of the starting population contains those cells, more preferably 10% or less, in one embodiment 5% or less. In other embodiments the population contains 4% or less, 3% or less, 2% or less, or 1% or less, respectively.

The starting population of cells can be isolated by known techniques such as selecting erythrocyte progenitor cells from adult hematopoietic tissue, bone marrow, peripheral blood, fetal liver cells, splenic tissue, umbilical cord blood, or umbilical cord tissue of an individual. The starting population of cells can be isolated from a mammal, including a human, rodent, pig, cat, primate, or dog.

One embodiment of the invention provides methods for determining the genotoxic effect of a test compound on a human erythroid cell. One can use any technique to obtain a population of cells that contains erythroid progenitors. For example, in one embodiment, the starting population is isolated by selecting for human cells which express glycophorin A and/or transferrin receptor. In one embodiment, the starting population of cells is isolated by selecting erythroid progenitors from a population of human cells which express at least two surface markers selected from the group consisting of CD34, CD41, CD71 and CD36. In one embodiment, the starting population of cells is a lineage marker negative (Lin−) population of human cells. In one embodiment, the starting population of cells consists of human cells which express CD34.

The invention provides methods to isolate the starting population of cells from human bone marrow. In one embodiment, these cells are isolated from human bone marrow from the blast region, that is, cells with high forward-scatter properties and low side-scatter properties as assessed by flow cytometry, which express GlyA and CD71. In another embodiment, the starting population of cells is isolated from human bone marrow by selecting for cells which express cell surface markers recognized by the 5F1 antibody and the CLB-Ery-3+ antibody, and do not express Gly-A. In yet another embodiment, the starting population of cells is isolated from human bone marrow by selecting for cells which express CD34.

The invention provides methods to isolate the starting population of cells from human umbilical cord blood or human umbilical cord tissue. In one embodiment, the starting population of cells is isolated from human umbilical cord or tissue by selecting for cells which express at least one of CD34, CD41, and HLA-DR, in another embodiment at least two of the markers and in another embodiment all three markers. In another embodiment, the starting population of cells is isolated from human umbilical cord or tissue by selecting for cells which express CD71, CD36, and do not express GlyA. In another embodiment, the starting population of cells is isolated from human umbilical cord or tissue by selecting for cells which express CD34.

The invention provides methods to isolate the starting population of cells from human peripheral blood. In one embodiment, the starting population of cells is the mononuclear cell (MNC) fraction of human peripheral blood. In another embodiment, the MNC fraction is isolated from human peripheral blood by density gradient centrifugation. In one embodiment, the starting population of cells is isolated from G-CSF mobilized peripheral blood by selecting for cells which express CD34.

The invention provides methods to isolate the starting population of cells from a mammal such as a mouse or human, including from bone marrow. For example, the starting population of cells can be isolated from mouse tissue by selecting for cells which do not express Ter-119. In another embodiment, the starting population of cells is isolated from mouse tissue by selecting for cells that do not express at least one of the cell surface markers selected from the group consisting of Lin, Sca-1, IL7-Rα, and CD41. In another embodiment, the starting population of cells is isolated from tissue such as mouse tissue by selecting for cells which express c-Kit and CD71. In another embodiment, the starting population of mouse cells is a lineage marker negative (Lin) population.

The starting population of cells of the invention can be isolated by an immunomagnetic technique or a flowcytometric technique. One or more antibodies can be used to isolate the starting population of cells. In one embodiment, the antibody recognizes a cell surface marker.

The methods of the invention provide culturing the starting population, which contains erythroid progenitor cells, for a sufficient time and under sufficient conditions to obtain erythropoietic growth. One can obtain the cells from the primary tissue of an animal, e.g. human. Although the technology to induce terminal differentiation of a population of cells from such tissue can take longer, it permits an in vitro assay system that is closer to real life. In one embodiment, the cells are cultured in an initial culture medium which enhances proliferation of the starting population of cells. The initial culture medium can be a medium which enhances proliferation of the starting population of cells. In one embodiment, the test compound is added to the culture medium after the cells are initially placed in culture. In one embodiment, the test compound can be added during terminal differentiation. For example, 0-12 hours after mouse cells are placed in culture, however longer time periods are also permissible such as 18-36 hours, including intervals such as 23, 24, 30. In human cells one typically waits longer, in some cell cultures up to 18 days. This can readily be determined based upon the cell type used. For example, in one embodiment, the test compound is added to the culture medium for 12-24 hours. Other embodiments include 18-48 hours. In one embodiment the cells are washed to remove the test compound and fresh culture medium is added, also referred to as an erythroid differentiation culture medium. The fresh culture medium can promote the erythroid differentiation of the erythroid progenitor cells into terminally differentiated erythrocytes.

The culture media for use in the invention includes any medium which supports erythropoietic growth. In one embodiment, the culture medium is a minimal culture medium. One embodiment of the invention provides an erythroid differentiation culture medium comprising Iscove's Modified Dulbecco Medium (IMDM), 20% fetal bovine serum, 2 mM glutamine, and 0.1 mM β-mercaptoethanol. In one embodiment, the initial culture medium comprises Iscove's Modified Dulbecco Medium (IMDM), 15% (fetal bovine) serum, 1% detoxified bovine serum albumin (BSA), 200 μg/ml holo-transferrin, 10 μg/ml insulin, 2 mM glutamine, 0.1 mM β-mercaptoethanol, 5 U/ml erythropoietin, 100 ng/ml stem cell factor (SCF), and 10 μM dexamethasone. In another embodiment, the initial culture medium comprises Iscove's Modified Dulbecco Medium (IMDM), 15% (fetal bovine) serum, 1% detoxified bovine serum albumin (BSA), 200 μg/ml holo-transferrin, 10[g/ml insulin, 2 mM glutamine, 0.1 mM β-mercaptoethanol, 10 U/ml erythropoietin, 100 μg/ml stem cell factor (SCF), and 10 μM dexamethasone.

One embodiment of the invention provides culturing the starting population of cells on a surface comprising fibronectin. For example, the surface can comprises 2 μg/cm² fibronectin.

One embodiment of the invention provides culturing the cells under hypoxic conditions. For example, the hypoxic conditions can comprise 5-15, e.g., 3-10%, e.g., about 5-10% 0₂, 5% C0₂, and balance N₂.

The methods of the invention can be used to determine the genotoxic effect of any substance to which a human may be exposed. Test compounds include pharmaceuticals, diagnostics, pesticides, cosmetics, vaccines, lotions, foods and packing materials. In one embodiment, the test compound is a therapeutic compound or a diagnostic compound. In another embodiment, the test compound is a candidate compound for use in treating an erythropoietic developmental defect, and the number of MN-PCEs and the number, size, and shape of PCEs are compared between healthy erythropoietic cultures and erythropoietic cultures exhibiting an erythropoietic developmental disorder, in both the presence and absence of the test compound. Examples of erythropoietic developmental defects include but are not limited to sickle cell anemia, thalassemias, polycythemia vera, and other myeloproliferative disorders. The test compound can be added to the cells at a concentration which is not cytotoxic to the cells. Genotoxic effects which can be determined using the present invention include clastogenetic effects and aneugenetic effects. In one embodiment, the test compound is metabolically activated before it is added to the starting population of cells, including by incubation with liver microsomes or a hepatocyte culture.

Any method which detects MN can be used in the methods of the invention. For example, the percentage of cells comprising micronuclei can be determined using flow cytometry, histological analysis and scoring (e.g. via histological examination and differential counting), automated image analysis platforms, and biochemical methods, including analysis of the ratio of DNA content to hemoglobin content.

The invention also provides methods for screening a group of test compounds to determine the genotoxic effect of each individual test compound on erythroid cells, by selecting at least four individual test compounds to comprise the group of test compounds; and determining the genotoxic effect of each individual test compound on an erythroid cell using the methods of the invention. These high throughput methods include screening a group of test compounds simultaneously in a series of parallel cultures. In one embodiment, the group of test compounds comprises at least 30 different individual test compounds. In another embodiment, the group of test compounds comprises at least 300 different individual test compounds. In yet another embodiment, the group of test compounds comprises at least 3000 different individual test compounds. In one embodiment, the genotoxic effect of a test compound is determined at multiple concentrations for that compound, including for example at least 5 different concentrations, or at least 25 different concentrations. These methods provide for an in vitro screen that more accurately reflect in vivo response, and hence, can predict clinical outcome more accurately and/or supply than most current screens.

### BRIEF DESCRIPTION OF THE DRAWINGS

FIG. 1 is a micrograph of a purified bone marrow (BM) sample that has been stained with acridine orange (AO) and visualized using fluorescence microscopy (adapted from Krishna et al., 2000). Shown in the image are nucleated cells (green/yellow), NCEs (khaki/green because they are mostly devoid of nucleic acid), and PCEs (bright orange/red mostly due to the presence of ribosomes and mRNA). Also shown is a MN-PCE, which is a newly-formed erythrocyte that contains the remnants of prior genetic damage, either clastogenic or aneugenic, and which is “micronulceated” as a consequence of this damage.

FIGS. 2A-2C illustrate the invention. FIG. 2A is an illustration of the standard in vivo MN assay. FIG. 2B is an illustration of the present invention conducted in vitro using a hematopoietic population. FIG. 2C is an illustration of the invention conducted in vitro using a human hematopoietic population.

FIG. 3 is an illustration of a flow cytometric technique that can be used to track the progress of erythropoietic growth in culture. Erythropoietic growth was induced from Ter-119⁻ fetal liver (FL) over a culture period of two days. The abscissa and ordinate on these density plots both represent log-scale relative fluorescence; the former provides a measure of Ter-119 expression while the latter provides a measure of CD71 expression. As erythropoietic growth occurs in vitro, a population that is initially devoid of Ter-119, an erythroid-specific cell surface protein, begins to express Ter-119 after first increasing its expression of CD71, the transferrin receptor. In terms of the flow cytometric regions drawn in these plots, the cells progress from stages R1 and R2 into stages R3, R4, and finally R5 as they differentiate into erythrocytes. Also shown in FIG. 3 are histological samples from these cultures at day 1 and day 2 that have been stained with benzidine-giemsa stain; yellow coloration in the day 2 population indicates that the cells are benzidine positive and, consequently, that the cells contain hemoglobin. The scale-bar in these images is 20 μm. Juxtaposed with these flow cytometric and histological images is an artist's rendition of erythropoietic growth, with each stage of differentiation labeled with the flow cytometric region that it approximately corresponds to (Lodish 2003; Zhang, Socolovsky et al. 2003).

FIG. 4 shows the differentiation profile of Lin− bone marrow, a progenitor-rich population of cells, cultured in vitro for 3 days on fibronectin-coated plates exposed to Epo for the first day of culture. Cultured cells were examined by both flow cytometry and benzidine-Giemsa stain after each day in culture. Shown are density plots for whole bone marrow and for Lin⁻ progenitor-rich populations on day 0, 1, 2, and 3 of growth are shown. Also shown are representative micrographs of benzidine-Giemsa stains of the harvested populations. The arrowhead indicates a hemoglobin⁺ normoblast, and the arrow indicates an enucleated reticulocyte. Scale bars: 20 μm.

FIG. 5 is micrograph that shows a representative field of a population following erythropoietic culture. Lin⁻ mouse BM from C57BL/6J mice, aged 6-8 weeks, were cultured under condition #7 (defined in Table I), and stained with AO.

FIG. 6 is a graph of the dose-response to BCNU as measured in C57BL/6J mice by the standard in vivo MN assay. BCNU, a known genotoxicant that has been shown to form interstrand cross-links and that is known to behave as a clastogen, was administered by IP injection and animals were sacrificed 24 hours prior later. The MN frequency in the PCE population increased with increasing BCNU dose up to the maximum dose tested (10.5 mg/kg), illustrating the ability of the in vivo MN assay to detect dose-dependent genotoxicity.

FIG. 7 is a graph of the dose-response to BCNU as measured by conducting the present invention using the Lin− fraction of C57BL/6J (mouse) BM as an initial population.

FIGS. 8A and 8B show plots of the dose-response to BCNU as measured by counting cell numbers from dosed cultures and then plotting these as relative values of untreated (control culture) cell numbers. The figure provides data using two different vehicles; data for ethanol is shown in FIG. 8A and data for dimethyl sulfoxide is shown in FIG. 8B. Cell numbers, relative to those from untreated cultures, are presented both for total cells and for erythroid specific cells.

FIGS. 9A-9D show another example of detection of genotoxicity through in vitro erythropoiesis. FIG. 9A are representative micrographs from both treated and untreated cultures. The arrow indicates a normally enucleated PCE and the arrowheads indicate micronucleated PCEs. Scale bars: 20 μm. Shown in FIG. 9B are graphical representations of the response of this culture system to BCNU quantified in terms of growth-inhibition and micronucleus-induction. In the left-hand plot, fractional survival is plotted vs. BCNU concentration. In the right-hand plot, micronucleus frequency is plotted vs. BCNU concentration. Shown in FIG. 9C are graphical representations of the response of this culture system to MNNG quantified in terms of growth-inhibition and micronucleus-induction. Shown in FIG. 9D are graphical representations of the response of this culture system to MMS quantified in terms of growth-inhibition and micronucleus-induction. Data are presented as the mean of 3 independent cultures +/− the standard deviation. * indicates a significant difference (P<0.05) from untreated control cultures as determined by the two-tailed t test. ** indicates a significant difference (P<0.01) from the untreated control cultures as determined by the two-tailed t test. **** indicates a significant difference (P<0.0001) from the untreated control cultures as determined by the two-tailed t test.

FIGS. 10A-10D show flow cytometry and benzidine-Giemsa histology data generated by analysis of populations of cells harvested for FIG. 9. This data demonstrates that in vitro erythropoiesis continues after treatment with alkylating agents. The arrowheads indicate hemoglobin⁺ cells containing micronuclei. Scale bars: 20 μm.

FIG. 11 is a graph which shows the results of a series of experiments. Lin-bone marrow cells were cultured in various atmospheric conditions, as indicated in the legend, while the numbers (1-16) correspond to the culture conditions detailed in Table I. Upon harvest of each culture, a total cell count was taken and representative slides were prepared and stained to provide estimates of the fraction of the harvested population that consisted of PCEs. Over 2000 cells were visualized and counted per slide in order to estimate the PCE fraction in the total population. This data is also part of FIG. 14a.

FIG. 12 shows the results from numerical modeling conducted to estimate the primary and secondary effects of the independent parameters that were studied in the experiment that is described by FIG. 11. Note, this analysis was performed in two different manners. The results of the second analysis are presented in FIG. 16.

FIG. 13 is a bar graph of data obtained from experiments performed to determined the effect of Epo on the erythropoietic growth of Lin− bone marrow cells. Lin⁻ bone marrow cells were cultured for 1 day in media containing serum along with Epo at variable concentrations. Epo was removed from the culture media at the end of 1 day, and culture was continued for 2 more days in media with serum. At the end of the third day, the resulting populations were removed from culture and erythropoietic growth was quantified as the product of total cell counts and flow cytometric region fractions. Specifically, erythroid-specific growth at two late stages was calculated for each culture by taking the product of the total cell count and the fraction of the population found to have the indicated surface phenotype (determined by flow cytometry). These erythroid growth values were then normalized to the number of Lin⁻ cells seeded. Data are presented as the mean from 3 independent cultures +/− the standard deviation. ** indicates a significant difference (P<0.01) from standard culture conditions (2 U/mL Epo) as determined by the two-tailed t test.

FIGS. 14A is a bar graph and FIG. 14B are micrographs demonstrating data obtained from experiments designed to provide an estimation of assay throughput and the relative erythropoietic growth effects of various physiologic stimuli, specifically focusing on development of experimental design and measured growth. (FIG. 14A) Lin⁻ bone marrow cells were cultured for 1 day in media containing serum and various combinations of soluble growth factors as listed on the abscissa; these specific 6-factor combinations constitute an orthogonal fractional-factorial design (also represented in Table 1). After 1 day, all soluble erythropoietic growth factors were removed and the cells were cultured for either 2 or 3 additional days in medium with serum. At harvest, the cells were removed from culture and late erythropoietic growth was quantified. For each culture, PCE numbers were calculated by taking the product of the total cell count and the PCE fraction of the population as determined by differential cell counting after staining with acridine orange (>2000 cells scored per culture). Growth calculations were normalized to input cell numbers and are presented as the mean of 3 independent cultures +/− the standard deviation. (FIG. 14B) Culture methods that yielded similar populations at harvest were grouped into subsets (α, β, γ, and δ); growth characteristics for these subsets were quantified and representative micrographs were acquired. It was found that cultures not exposed to Epo or SCF (subset α) underwent no appreciable growth and contained less than 3% PCEs. Cultures exposed to SCF but not Epo (subset γ) expanded approximately 2.5-fold in total cell numbers, but again contained less than 3% PCEs. Cultures exposed to both Epo and SCF (subset β) expanded approximately 13-fold in total cell numbers and yielded populations in which approximately 31% of all cells are PCEs. Finally, cultures exposed to both Epo and Dex but not SCF (subset δ) expanded approximately 4-fold while producing populations in which approximately 51% of all cells are PCEs. Growth calculations are presented as the mean of all cultures in a subset ± the standard deviation. Scale bars: 20 μm.

FIG. 15 has several data plots of results from flow cytometry experiments and micrographs of benzidine-Giemsa stained cells, providing a dynamic analysis of Lin⁻ bone marrow cultured for 3 days under improved erythropoieitic conditions. Purified Lin⁻ cells were cultured in vitro for 3 days on fibronectin-coated plates in medium containing serum. Epo, SCF, Dex, and IGF-I were included in the medium for the first day of culture, and then the medium was changed to remove soluble growth factors. The differentiation profile of the cultured cells was examined by both flow cytometry and benzidine-Giemsa stain after each day in culture. After the third day, flow cytometry indicated that much of the cultured population expressed Ter-119 and CD71. Furthermore, benizidine-Giemsa stain revealed that many cells in the harvested population were enucleated and expressing hemoglobin. The arrowhead indicates a hemoglobin⁺ normoblast, and the arrow indicates an enucleated reticulocyte. Scale bars: 20 μm.

FIGS. 16A and 16B contain two graphical representations of data from experiments estimating assay throughput and the relative erythropoietic growth effects of various physiologic stimuli, shown as growth prediction by multi-linear regression and scaled parameter estimates. Least-squares regression was used to generate the linear model that best predicts erythropoietic growth from the experimental design matrix. (FIG. 16A) The calculated growth responses from each culture (data points) are plotted vs. the response predicted by the regression model (abscissa/solid line). The regression model is capable of predicting PCE-growth in this culture system (R²=0.95) from information about the key culture parameters listed in FIG. 16B. (FIG. 16B) The scaled parameter estimates that constitute the regression model are given. The parameters are listed in order of decreasing impact on erythropoietic growth, with the most significant growth factor (Epo) at the top. The error bars indicate the span of the 95% confidence intervals for the parameter estimates.

FIGS. 17A-17C contain three bar graphs and FIG. 17D contains two micrographs which are graphical representations of data from experiments that show MGMT expression affects both the frequency and the dynamics of micronucleated reticulocyte formation in the bone marrow following in vivo exposure to BCNU. Wild-type (C57BL/6J) and transgenic (MGMT^{-/-} on C57BL/6J background) male mice aged 6-8 weeks were dosed with either BCNU or vehicle control (10% EtOH in PBS) by intraperitoneal injection. After either 24 h, 48 h, or 72 h, the dosed animals were sacrificed, the bone marrow was flushed from the femurs, and slides were prepared and stained with acridine orange for differential cell counting. (FIG. 17) The micronucleus frequency in bone marrow PCEs (reticulocytes) was quantified 24 h after exposure to BCNU, and MGMT^{-/-} mice were found to have significantly fewer microncucleated PCEs after exposure to BCNU at doses of 3.5 and 7.0 mg/kg. * indicates a significant difference (P<0.05) from MGMT+/+mice as determined by the two-tailed t test. (FIG. 17B) The micronucleus frequency in bone marrow PCEs was quantified 48 h after exposure to BCNU, and MGMT^{-/-} mice were found to have significantly more microncucleated PCEs after exposure to BCNU at doses of 3.5 and 7.0 mg/kg. *** indicates a significant difference (P<0.001) from the MGMT^{+/+} mice as determined by the two-tailed t test. (FIG. 17C) The micronucleus frequency in bone marrow PCEs was quantified 72 h after exposure to BCNU, and MGMT^{-/-} mice were found to have more microncucleated PCEs after exposure to BCNU at doses of 3.5 and 7.0 mg/kg. Sample size is currently being increased to determine significance. (FIG. 17D) Representative micrographs are shown for MGMT^{-/-} BM both 48 h and 72 h after exposure to BCNU (at 7 mg/kg). 72 h after exposure to BCNU, the PCE:NCE ratio has decreased dramatically, indicating that erythroid progenitors in MGMT^{-/-} BM that are approximately 3 d away from becoming reticulocytes exhibit profound cytotoxic sensitivity to BCNU exposure, whereas those that are 48 h away from reticulocyte formation exhibit less cytotoxic sensitivity. In MGMT^{+/+} BM samples, the PCE:NCE ratio remained constant at various times after BCNU exposure (at 7 mg/kg, data not shown). Differential cell counts are currently being conducted to quantify these effects and assess statistical significance.

FIG. 18 has several data plots of results from flow cytometry experiments and micrographs of benzidine-Giemsa stained cells, which provide a dynamic analysis of MGMT^{-/-} Lin⁻ bone marrow cultured for 3 days under improved erythropoieitic conditions. Purified Lin⁻ cells from MGMT^{-/-} mice were cultured in vitro for 3 days on fibronectin-coated plates in medium containing serum. Epo, SCF, Dex, and IGF-I were included in the medium for the first day of culture, and then the medium was changed to remove soluble growth factors. The differentiation profile of the cultured cells was examined by both flow cytometry and benzidine-Giemsa stain after each day in culture. After the third day, flow cytometry indicated that much of the cultured population had acquired a late erythroid surface phenotype during culture. Furthermore, benizidine-Giemsa stain revealed that many cells in the harvested population were enucleated and expressing hemoglobin. The arrowhead indicates a hemoglobin⁺ normoblast, and the arrow indicates an enucleated reticulocyte. Scale bars: 20 μm.

FIGS. 19A and 19B contain graphical representations of experimental data which indicate that Lin⁻ bone marrow from MGMT^{-/-} mice is more sensitive to growth-inhibition and MN-formation than Lin⁻ bone marrow from MGMT^{+/+} mice when treated with BCNU during erythropoietic culture. Purified Lin⁻ cells from MGMT^{-/-} and MGMT^{+/+} mice were cultured in vitro for 1 day on fibronectin-coated plates in medium containing serum, Epo and other erythropoietic growth factors (SCF, Dex, and IGF-I). After one day, the medium was changed to a minimal formulation containing serum without erythroid-specific cytokines. Populations were then cultured for 2 additional days before harvest at 72 h. BCNU was introduced into the culture media at various times (10 h, 23 h, or 30 h) after seeding. At harvest, the cells were removed from culture, and genotoxic effects were quantified through viable cell counts and micronucleus enumeration. The fractional survival of cultures after treatment was determined by normalizing the mean of viable cell counts from those cultures to the mean of viable cell counts from untreated cultures. The micronucleus frequency in cultures was determined by acridine orange stain and differential cell counting (>2000 PCEs scored per culture). (FIG. 19A) The MN response of Lin⁻ bone marrow from MGMT^{+/+} and MGMT^{-/-} mice to BCNU in this erythropoietic culture system is quantified. Micronucleus frequency is plotted vs. BCNU concentration. + indicates a significant difference (P<0.05) between MGMT^{+/+} and MGMT^{-/-} cultures as determined by the two-tailed t test. ++ indicates a significant difference (P<0.01) between MGMT^{+/+} and MGMT^{-/-} cultures as determined by the two-tailed t test. * indicates a significant difference (P<0.05) from the vehicle control as determined by the two-tailed t test. ** indicates a significant difference (P<0.01) from the vehicle control as determined by the two-tailed t test. * * * indicates a significant difference (P<0.001) from the vehicle control as determined by the two-tailed t test. (FIG. 19B) The growth-inhibition of Lin⁻ bone marrow from MGMT^{+/+} and MGMT^{-/-} mice by BCNU in this erythropoietic culture system is quantified. Fractional survival is plotted vs. BCNU concentration. (FIG. 19C) This model is a representation of erythropoietic growth and responses to genotoxic exposure as observed in this culture system. Key receptors for normal erythropoiesis and markers of late-erythroid cells are indicated. Based on FIG. 19a and FIG. 19b, genotoxic exposure of primitive cells (late BFU-Es and CFU-Es) is likely to stimulate apoptosis, whereas genotoxic exposure of later erythroid cells is likely to result in the growth of micronucleated reticulocytes.

### DETAILED DESCRIPTION OF THE INVENTION

Aspects of the present invention relate to a method for determining the genotoxic effect of a test compound on an erythroid cell. The method comprises culturing in vitro a starting population of cells, wherein said population of cells contains erythroid progenitors, for a sufficient time and under sufficient conditions to obtain erythropoietic growth, adding a test compound to the culture medium, and harvesting the differentiated erythroid populations, and then measuring at least one of the following characteristics: (i) total number and presence of micronuclei (MN) in the PCEs, wherein the presence of greater level of MN in the cells relative to a control population of cells not exposed to the test compound indicates the genotoxic effect of said test compound; (ii) total cell number of erythroid-specific cells, wherein a decrease in total cell number of erythroid-specific cell numbers provides and indication of a general cytotoxic effect of said test compound; and (iii) PCE number, size or shape, wherein a change in PCE number, size, or shape provides an indication of the efficacy of said test compound in treating a given erythropoietic defect. In one embodiment of the invention, the erythroid progenitor is a cell that can be induced to undergo erythropoiesis in vitro. In another embodiment, the erythroid progenitor is from the group consisting of cells near the colony-forming unit erythroid (CFU-E) stage of erythropoiesis, the burst-forming unit erythroid (BFU-E) stage of erythropoiesis, the CFU-granulocyte erythroid macrophage megakarocyte (CFU-GEMM) stage of erythropoiesis, or long-term repopulating hematopoietic stem cell (LT-HSC), or combinations thereof. In another embodiment, the starting population of cells is substantially free of mature granulocytes, reticulocytes, macrophages, T cells, B cells, and erythrocytes. In another embodiment, the starting population of cells is substantially free of differentiated erythrocytes. In another embodiment, the starting population of cells is isolated by selecting erythrocyte precursor cells from a population of cells selected from the group consisting of adult hematopoietic tissue, bone marrow, peripheral blood, fetal liver cells, splenic tissue, umbilical cord blood, and umbilical cord tissue of an individual.

In one embodiment the starting population of cells is isolated from a mammal. In one embodiment the mammal is a human. In another embodiment, the starting population of cells isolated from a mammal are isolated by immunoselection for expression of glycophorin A and/or transferrin receptor. In another embodiment, the starting population of cells isolated from a human is isolated by selecting erythroid progenitors from a population of human cells which express at least two of the following surface markers: CD34, CD41, CD71 and CD36. In another embodiment, the starting population of cells is a lineage marker negative (Lin−) population of human cells. In another embodiment, the starting population of cells are human cells which express CD34. In another embodiment, the starrting population of cells is isolated from human bone marrow. In another embodiment, the starting population of cells isoloated from human bone marrow is by selecting for cells with high forward scatter properties and low side scatter properties and which express GlyA and CD71. In another embodiment the starting population of cells isoloated from human bone marrow is isolated by selecting for cells which express cell surface markers recognized by the SF1 antibody and the CLB-Ery-3+ antibody, and do not express Gly-A. In another embodiment, the starting population of cells isoloated from human bone marrow is isolated by selecting for cells which express CD34+. In another embodiment the starting population of cells is isolated from human umbilical cord blood or human umbilical cord tissue. In another embodiment, the starting population of cells is isolated by selecting for cells which express CD34, CD41, and HLA-DR. In another embodiment, the starting population of cells isolated from human umbilical cord blood or human umbilical cord tissue is isolated by selecting for cells which express CD71, CD36, and do not express GlyA. In another embodiment, the starting population of cells isolated from human umbilical cord blood or human umbilical cord tissue is isolated by selecting for cells which express CD34. In another embodiment the starting population of cells is isolated from human peripheral blood. In another embodiment the starting population of cells is the mononuclear cell (MNC) fraction of human peripheral blood. In another embodiment, the MNC fraction of human peripheral blood is isolated by density gradient centrifugation. In another embodiment the starting population of cells isolated from human peripheral blood is isolated from G-CSF mobilized peripheral blood by selecting for cells which express CD34.

In another embodiment the starting population of cells is isolated from a mouse. In another embodiment the starting population of cells is isolated from mouse bone marrow. In another embodiment the starting population of cells isolated from mouse bone marrow does not express at least one of the cell surface markers selected from the group consisting of Gr-1, Mac-1, CD3, B-220, and Ter-119. In another embodiment, the starting population of cells isolated from mouse bone marrow does not express at least one of cell surface markers Lin, Sca-1, IL7-Rα, or CD41. In another embodiment the starting population of cells isolated from mouse bone marrow which does not express at least one of cell surface markers Lin, Sca-1, IL7-Rα, or CD41, expresses c-Kit and CD71. In another embodiment the starting population of cells isolated from mouse bone marrow is a lineage marker negative (Lin−) population. In another embodiment the starting population of cells isolated from mouse bone marrow does not express Ter-119. In another embodiment the starting population of cells is isolated by an immunomagnetic technique or a flowcytometric technique.

In one embodiment of the invention one or more antibodies is used in the method for determining the genotoxic effect of a test compound on an erythroid cell to isolate the starting population of cells using an immunomagnetic technique or a flowcytometric technique. In another embodiment the antibody used to isolate the starting population of cells using an immunomagnetic technique or a flowcytometric technique recognizes a cell surface marker.

In another embodiment, the starting population of cells is cultured in an initial culture medium which enhances proliferation of the starting population of cells. In one embodiment, the initial culture medium includes an additional factor that is erythropoietin, holotransferrin, dexamethasone, stem cell factor, insulin, or IGF-1.

In one embodiment, the test compound is added to the culture medium for 12-24 hours, after which the cells are washed to remove the test compound and fresh culture medium is added. In one embodiment the fresh culture medium that is added promotes the erythroid differentiation of the erythroid progenitor cells into terminally differentiated erythrocytes (erythroid differentiation culture medium). In another embodiment, the added erythroid differentiation culture medium is a minimal culture medium. In one embodiment the erythroid differentiation culture medium contains IMDM, 20% fetal bovine serum, 2 mM glutamine, and 0.1 mM β-mercaptoethanol. In another embodiment, the initial culture medium that enhances proliferation of the starting population of cells contains Iscove's Modified Dulbecco Medium (IMDM), 15% (fetal bovine) serum, 1% detoxified bovine serum albumin (BSA), 200 μg/ml holo-transferrin, 10 μg/ml insulin, 2 mM glutamine, 0.1 mM β-mercaptoethanol, 10 U/ml erythropoietin, 100 μg/ml stem cell factor (SCF), and 10 μM dexamethasone.

In one embodiment of the invention, the starting population of cells is cultured on a surface comprising fibronectin. In another embodiment the surface comprises about 2 μg/cm² fibronectin.

In another embodiment of the invention the starting population of cells are cultured in hypoxic conditions. In one embodiment the hypoxic conditions comprise 3-15% 0₂, about 5% C0₂, and N₂.

In another embodiment of the invention the test compound is any compound to which a human can be exposed. In one embodiment, the test compound is a pharmaceutical, diagnostic, pesticide, cosmetic, vaccine, lotion, food, agro-chemical, nanoparticle, commodity chemical, chemical intermediate, biomaterial or a packing material. In one embodiment the test compound is a therapeutic compound or a diagnostic compound. In another embodiment, the therapeutic compound or diagnostic compound test compound is a candidate compound for use in treating an erythropoietic developmental defect, and the number of MN-PCEs and the number, size, and shape of PCEs are compared between control cultures and cultures exhibiting an erythropoietic developmental disorder. In one embodiment, the erythropoietic developmental defect is sickle cell anemia, thalassemias, polycythemia vera, or other myeloproliferative disorder.

In one embodiment the test compound is added to the culture medium at a concentration which is not cytotoxic to the cells.

In another embodiment, the genotoxic effect is a clastogenetic effect or an aneugenetic effect.

In another embodiment the test compound is metabolically activated before it is added to the starting population of cells. In one embodiment the test compound is metabolically activated by incubation with liver microsomes or a hepatocyte culture.

In another embodiment of the invention the percentage of cells comprising micronuclei is determined by the method of flow cytometry, histological analysis and scoring, automated image analysis platforms, or biochemical analyses.

Another aspect of the invention relates to a method for screening a group of test compounds to determine the genotoxic effect of each individual test compound on erythroid cells. The method comprises selecting at least four individual test compounds to comprise the group of test compounds, and determining the genotoxic effect of each individual test compound on an erythroid cell using the above described method for determining the genotoxic effect of a test compound on an erythroid cell. Any and all embodiments herein described for the method for determining the genotoxic effect of a test compound on an erythroid cell can be applied to the a method for screening a group of test compounds to determine the genotoxic effect of each individual test compound on erythroid cells. In one embodiment the group of test compounds is screened simultaneously in a series of parallel cultures. In one embodiment the group of test compounds comprises at least 30 different individual test compounds. In one embodiment, the group of test compounds comprises at least 300 different individual test compounds. In one embodiment the group of test compounds comprises at least 3000 different individual test compounds.

In another embodiment each genotoxic effect of an individual test compound is determined in the method for screening a group of test compounds, at multiple concentrations for that compound. In one embodiment the genotoxic effect of an individual test compound is determined for at least 5 different concentrations. In another embodiment the genotoxic effect of an individual test compound is determined for at least 25 different concentrations.

In one embodiment, the present invention is directed to novel methods to assay the genotoxic effects of compounds on human hematopoietic cells such as erythroid cells in vitro, by, for example, culturing a starting population of cells enriched for colony-forming units erythroid (CFU-Es) or other late-erythroid cells such as burst-forming units erythroid (BFU-E's) or normoblasts, exposing the cells to a test compound, inducing differentiation of the CFU-Es or later-erythroid cells into erythrocytes, and detecting the presence of micronuclei in the final population comprising erythrocytes. The frequency of micronuclei in the fully differentiated erythrocytes and changes in the number, size and shape of PCEs can indicate the genotoxic effects of the test compound, providing a measure of the compound's genotoxic risk to humans. The methods of the present invention can also be used to screen candidate therapeutic agents for the treatment of erythropoietic defects, including changes in the number, size and shape of PCEs that restore a normal phenotypic profile to a population that displays a disease phenotype profile in the absence of the agent. The invention also provides methods for using this culture system, along with automated analysis, to conduct a variety of high-throughput assays, including screening multiple dose-compound combinations for micronucleus induction effects.

Micronuclei

Micronuclei are typically membrane-bound, extra-nuclear, sub-2n DNA structures resulting from double-strand chromosome breaks or from the dysfumction of the mitotic spindle apparatus. Micronuclei, sometimes referred to herein as MN, are also known as Howell-Jolly bodies in the hematology literature.

There are at least four recognized mechanisms by which MNs can develop in poly-chromatic erythrocytes (PCEs): 1) loss of acentric fragments during mitosis, 2) chromosome breakage, 3) loss of entire chromosomes during mitosis, and 4) apoptosis (Heddle, Cimino et al. 1991). Therefore, detection of increased MN-PCE frequency over baseline levels is an indication that the test compound is either a genotoxin or a mitotic spindle poison.

Starting Population of Cells: Erythroid Progenitor Cells

In the first step of the method of the present invention, primary tissue is harvested to isolate a starting population of cells that is enriched in erythroid progenitor cells. Immunogenetic, flowcytometric, and other separation techniques can be used to isolate the starting population of cells which contains erythroid progenitor cells.

During erythropoiesis, cell differentiation along the erythroid lineage ultimately results in the formation of enucleated red blood cells. In humans, this differentiation occurs over a two week span. The earliest progenitor is a long-term repopulating hematopoietic stem cell (LT-HSC), another progenitor is the BFU-E (Burst-Forming Unit-Erythroid), which is small and without distinguishing histologic characteristics. The stage after the BFU-E is the CFU-E (Colony Forming Unit-Erythroid), which is larger than the BFU-E and immediately precedes the stage where hemoglobin production begins. The cells that begin producing hemoglobin are the immature erythrocytes, which not only begin producing hemoglobin, but also start condensing their nuclei to eventually become mature erythroblasts. The mature erythroblasts are smaller than the immature erythrocytes and have a tightly compacted nucleus which is expelled as the cells become reticulocytes. Reticulocytes are so named because these cells contain reticular networks of polyribosomes. As reticulocytes lose their polyribosomes and mitochondria, they become mature red blood cells (RBCs).

The starting population of cells is substantially free of mature granulocytes, reticulocytes, macrophages, T cells, B cells, and erythrocytes. For example, as discussed above, one selects for a cell type that does not express at least one cell surface marker selected from the group consisting of Lin, Sca-1, IL7-Rα, and CD41.

Preferably, the primary hematopoietic population is enriched for erythroid progenitors, which can be induced to differentiate into PCEs, in vitro, in the presence of a test compound, and it should not contain excessive PCEs. However, the erythropoietic culture of an unfractionated primary hematopoietic population will also result in the production of PCEs; therefore, any population containing erythroid progenitors, obtained from any species, can be used in the conduct of the present invention.

As used herein, erythroid progenitor refers to any cell that can be induced to undergo erythropoiesis in vivo. These erythroid progenitors will most preferably be near the CFU-E stage of erythropoiesis, but they may also be at an earlier stage of erythroid development, such as the burst-forming unit erythroid (BFU-E) stage of development, or at an even earlier stage of hematopoietic development such as the CFU-granulocyte erythroid macrophage megakaryocyte (CFU-GEMM or CFU-mix) developmental level. Any cell contained in the erythroid lineage, including the most primitive long-term repopulating hematopoietic stem cell (LT-HSC) and all intermediate cell types up to, but not including, the reticulocyte, or even a non-erythroid hematopoietic progenitor of sufficient plasticity to transdifferentiate, can be induced to yield PCEs and can be used in the present invention.

Cells near the CFU-E stage of erythropoiesis will often be preferred because they are at a stage of development that will provide a sufficient number of developmental divisions and, consequently, enough time for most test compounds to take effect, while still yielding PCEs within the next two to four days. Preferably, the starting population will be devoid of preexisting, enucleated PCEs. The presence of these cells in the initial population of short-term test cultures can confound test results because some PCEs that were formed in vivo, prior to introduction of a test agent, may still remain at time of harvest and analysis and may thus confound final cell counts.

Fully-differentiated hematopoietic cells of non-erythroid lineages, such as megakaryocytes, can not undergo erythropoietic growth, and the presence of these cells in the starting population may actually hinder the sensitivity and accuracy of test results. Culture of excessive non-erythroid cells may confound the results of this test system for three main reasons. First, such cells can deplete media supplements that support erythropoietic growth. Second, non-erythroid cells can act as unintended targets of test compounds and may blunt the compound's effect of on erythroid progenitor cells, which are the preferred target because they provide a metric of genotoxic damage in their MN-PCE progeny. For example, a blunting effect could arise from irreversible reactions between biomolecular components within non-erythroid cells and the active molecular components of the test agent and its derivatives. Third, apoptotic or necrotic decay of non-erythroid populations, which may not be supplied with necessary survival cues, can lead to excessive debris and “bystander” toxicity in a test culture. Depletion of initial hematopoietic tissue of such non-erythroid, differentiated cell types consequently leads to the enrichment of erythroid progenitor content mentioned above.

Obtaining Starting Population of Cells

The starting population of cells is obtained by selecting erythrocyte progenitor cells from adult hematopoietic tissue, bone marrow, peripheral blood, fetal liver cells, splenic tissue, umbilical cord blood, or umbilical cord tissue of an individual. The starting population of cells can be isolated from a mammal, including a human or a mouse.

The starting population of cells of the invention can be isolated by an immunoaffinity technique, including positive immunoselection, negative immunoselection, and immunomagnetic techniques, or a flowcytometric technique. One or more antibodies can be used to isolate the starting population of cells. In one embodiment, the antibody recognizes a cell surface marker. In another embodiment, the starting population can be isolated using methods based on changes in the adhesive properties of differentiating erythroid progenitors, including methods using activated substrates, beads, extracellular matrix, and extracellular matrix fragments.

Human Cell Populations

One embodiment of the invention provides methods for determining the genotoxic effect of a test compound on a human erythroid cell.

Human tissue types that can provide primary hematopoietic cell populations for the conduct of the present invention include bone marrow, peripheral blood, fetal liver, splenic tissue, and umbilical cord blood and tissue. When testing a therapeutic compound to be used in adults, the most preferable source of hematopoietic test populations is human BM, which is the normal site of adult erythropoiesis. This human BM can be genetically matched with the targeted epidemiological cohort in order to best predict clinical outcomes. Results of experiments detailed in the Examples section below demonstrate that the assay of the present invention is useful for determining genotype specific effects of a genotoxicant on cells used in the assay. Thus, hematopoietic cells which are obtained from a donor of a specific genotype will best reflect the genotoxicity on the cells of the particular donor or donor type. Much information is available in the literature regarding the isolation of erythroid progenitors from human BM, PB and cord blood.

The starting population can be isolated by selecting for human cells which express glycophorin A and/or transferrin receptor. In one embodiment, the starting population of cells is isolated by selecting erythroid progenitors from a population of human cells which express at least two surface markers selected from the group consisting of CD34, CD41, CD71 and CD36. In one embodiment, the starting population of cells is a lineage marker negative (Lin) population of human cells, also referred to as the Gr-1⁻, Mac-1⁻, CD3⁻, B-220⁻, Ter-119⁻ population. In one embodiment, the starting population of cells is at least 90% of human cells which express CD34, it can be all points in between, e.g. 95%, 98%, 99%, 99.8%.

The invention also provides methods to isolate the starting population of cells from human bone marrow. Phenotypic definitions of human BM subpopulations that have many of the preferred characteristics discussed above (enriched for CFU-Es and depleted of PCEs) are available. A BM subpopulation that shares side-scatter and forward-scatter characteristics with blood monocytes is comprised of immature cells of various lineages. Cells within this side-scatter, forward-scatter region contains nearly all of the marrow's colony-forming cells. This region is thus referred to as the “blast” region of the BM; it is also referred to herein as cells with high forward-scatter properties and low side-scatter properties as assessed by flow cytometry. Cells in this blast region can be subdivided based on expression of the glycophorin A (Gly A) cell surface protein and expression of the transferrin receptor (CD71), to obtain a population containing most of the CFU-Es in human BM. Accordingly, one population is the subpopulation of the BM blast region that expresses Gly A at an intermediate level and CD71 at a level in the high to intermediate range (Loken, Shah et al. 1987). In another embodiment, one can use a combination of three antibodies: 5F1, CLB-Ery-3, and VIE-G4. Two of these antibodies, 5F1 and CLB-Ery-3, bind specifically to antigens on CFU-Es and erythrocytes. The third antibody, an α-Gly-A antibody, termed VIE-G4, only binds to erythrocytes (Peschel, Konwalinka et al. 1987). In another embodiment, one can use the CD34 human hematopoietic stem and primitive progenitor cell marker to isolate the starting population of cells. CD34⁺ hematopoietic cells obtained using immunomagnetic techniques from human BM can be induced to undergo terminal erythropoiesis in culture (Giarratana, Kobari et al. 2005). Accordingly, in one embodiment, these cells are isolated from human bone marrow from the blast region which express GlyA and CD71. In another embodiment, the starting population of cells is isolated from human bone marrow by selecting for cells which express cell surface markers recognized by the 5F1 antibody and the CLB-Ery-3 antibody, and do not express Gly-A. In yet another embodiment, the starting population of cells is isolated from human bone marrow by selecting for cells which express CD34.

The invention provides methods to isolate the starting population of cells from human umbilical cord blood or human umbilical cord tissue. Many phenotypic characteristics of erythropoietic populations within human cord blood have been elucidated. A detailed analysis of cell surface antigen expression during erythropoiesis from cord blood mononuclear cells found that CD34, CD41, and human leukocyte antigen (HLA)-DR disappear as erythropoiesis progresses while CD71, CD36, and Gly A appear during erythropoietic differentiation (Okumura, Tsuji et al. 1992). This work indicates that CD71⁺, CD36⁺, Gly A⁻ mononuclear cells from human cord blood constitute a useful target population. Furthermore, it has been shown that the easily-isolated CD34⁺ hematopoietic cells in human cord blood can be induced to undergo terminal erythropoiesis in culture (Giarratana, Kobari et al. 2005). Accordingly, in one embodiment, the starting population of cells is isolated from human umbilical cord or tissue by selecting for cells which express CD34, CD41, and HLA-DR. In another embodiment, the starting population of cells is isolated from human umbilical cord or tissue by selecting for cells which express CD71, CD36, and do not express GlyA. In another embodiment, the starting population of cells is isolated from human umbilical cord or tissue by selecting for cells which express CD34.

The invention also provides methods to isolate the starting population of cells from human peripheral blood. Cell surface marker and physical characteristics of erythroid progenitors in human PB have also been identified, including markers for the isolation of these progenitors from PB (Sawada, Krantz et al. 1987). Erythroid progenitors are contained within the mononuclear cell (MNC) fraction of human PB, which is a population that can be isolated by density gradient centrifugation (Fibach and Rα chmilewitz 1993). These progenitors largely consist of BFU-Es which can be induced to yield CFU-Es in culture (Fibach et al.). It has also been demonstrated that isolating CD34⁺ hematopoietic cells from granulocyte colony-stimulating factor (G-CSF) mobilized PB provides a population that can be induced to undergo terminal erythropoiesis in culture (Giarratana, Kobari et al. 2005). Accordingly, in one embodiment, the starting population of cells is the mononuclear cell (MNC) fraction of human peripheral blood. In another embodiment, the MNC fraction is isolated from human peripheral blood by density gradient centrifugation. In one embodiment, the starting population of cells is isolated from G-CSF mobilized peripheral blood by selecting for cells which express CD34.

Murine Cell Populations

The invention provides methods to isolate the starting population of cells from a mouse, including from mouse bone marrow. In mouse hematopoietic populations, a detailed understanding of which surface phenotypes and physical characteristics correspond to various stages of erythroid development exists. For example, a BM subpopulation was recently identified that generates CFU-E colonies at an efficiency of approximately 70% (Terszowski, Waskow et al. 2004). The investigators who identified this population refer to this CFU-E population as the erythroid progenitor (EP) population. This EP population is characterized as having a surface protein phenotype that is Lin⁻, c-Kit⁺, Sca-1⁻, IL7-Rα⁻, IL-3Rα⁻, CD41⁻, and CD71⁺ and it comprises 0.41% of nucleated BM cells. In another embodiment, the starting population of cells is isolated from mouse tissue by selecting for cells that do not express at least one of the cell surface markers selected from the group consisting of Lin, Sca-1, IL7-Rα, and CD41, where Lin includes the markers Gr-1, Mac-1, CD3, B-220, and Ter-119. In another embodiment, the starting population of cells is isolated from mouse tissue by selecting for cells which express c-Kit and CD71. In another embodiment, the starting population of mouse cells is a lineage marker negative (Lin) population.

Depletion of BM of all Lin⁺ cells by immunomagnetic, flow cytometric, or other techniques, is another strategy to provide an initial erythropoietic target population for use in the present invention. Mouse fetal liver is highly erythropoietic and can also provide populations that will undergo terminal differentiation in liquid culture. In 10⁵ nucleated CD71^{med}, Ter-119⁻ cells from E14.5 fetal liver there are approximately 4.1×10⁴ CFU-Es, making this population a relatively pure starting population.

In one embodiment of the invention, the population of cells employed in this test is genetically matched with the targeted epidemiological cohort in order to predict clinical outcomes. These discussions with respect to human and murine cells are exemplary and can be adapted to any other species.

Culture Conditions

Any culture conditions which induce enucleation of erythroid progenitor cells, or their progeny, can be used.

The methods of the invention provide culturing the starting population, which contains erythroid progenitor cells, for a sufficient time and under sufficient conditions to obtain erythropoietic growth. In one embodiment, the cells are cultured in an initial culture medium which enhances erythropoietic proliferation of the starting population of cells. In one embodiment, the test compound is added to the culture medium after the cells are initially placed in culture. In one embodiment, the test compound is added to the culture medium 0-360 hours after the cells are initially placed in culture. The exact timing will depend upon the particular cells and the starting tissues. This can readily be determined. It may be useful to add the test compound to the cells while in the initial culture medium. When doing so, it may be useful to add the test compound to the cells at a latter stage of culture in this medium, e.g. at hour 23 of culture. Following sufficient exposure to the test compound, the test compound can be washed away, and/or fresh medium (e.g. erythroid differentiation culture medium) may be added. In some instances, it may not be necessary to wash the cells. In one embodiment, the test compound is added to the culture medium for 12-24 hours, after which the cells are washed to remove the test compound and fresh culture medium is added, also referred to as an erythroid differentiation culture medium. In another embodiment, the test compound is added to the culture medium at 18-48 hours, such as 23, 24, and 30 hours and all other points within. The fresh culture medium can promote the erythroid differentiation of the erythroid progenitor cells into terminally differentiated erythrocytes.

Human cells typically take longer than mouse cells to reach the appropriate stage for test compound addition. In one embodiment, one determines the stage of cellular progression for the specific cell type and adding the test compound at the appropriate time, e.g. adding the test compound when the cells are near the CFU-E stage or erythroblast (e.g., normoblast) stage.

The culture media for use in the invention includes any medium which supports erythropoietic growth, including enucleation of erythroid progenitor cells. The culture medium may contain additional factors (e.g. cytokines or other growth promoting factors) which have been identified to enhance erythropoietic growth. The term “additional factors” as used herein in this context is meant to reflect addition above amounts which may be present in a serum component of the medium. Such factors include, without limitation, erythropoietin, holotransferrin, dexamethasone, stem cell factor, insulin, IGF-1. One or more, and any combination of such factors may be used. The combination and concentrations of these factors may be determined and optimized for each particular cell type and/or assay. Such factors are preferably added to the cells in the initial culture medium, and may be removed from the cells at the appropriate time (e.g. the factors are not present in the erythroid differentiation culture medium).

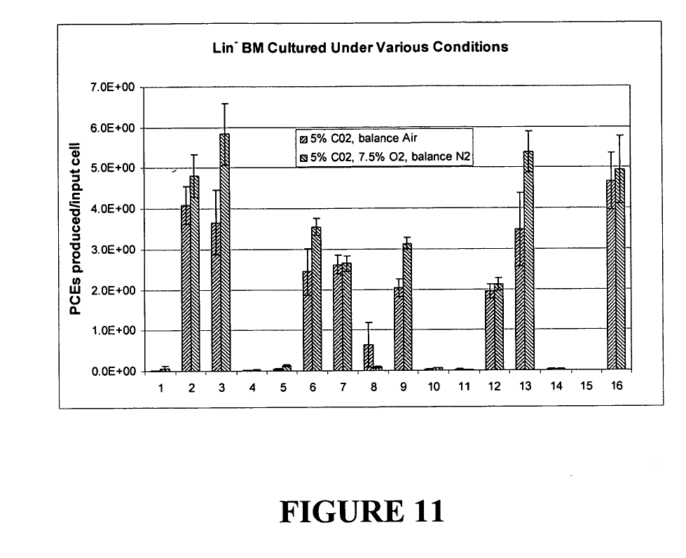

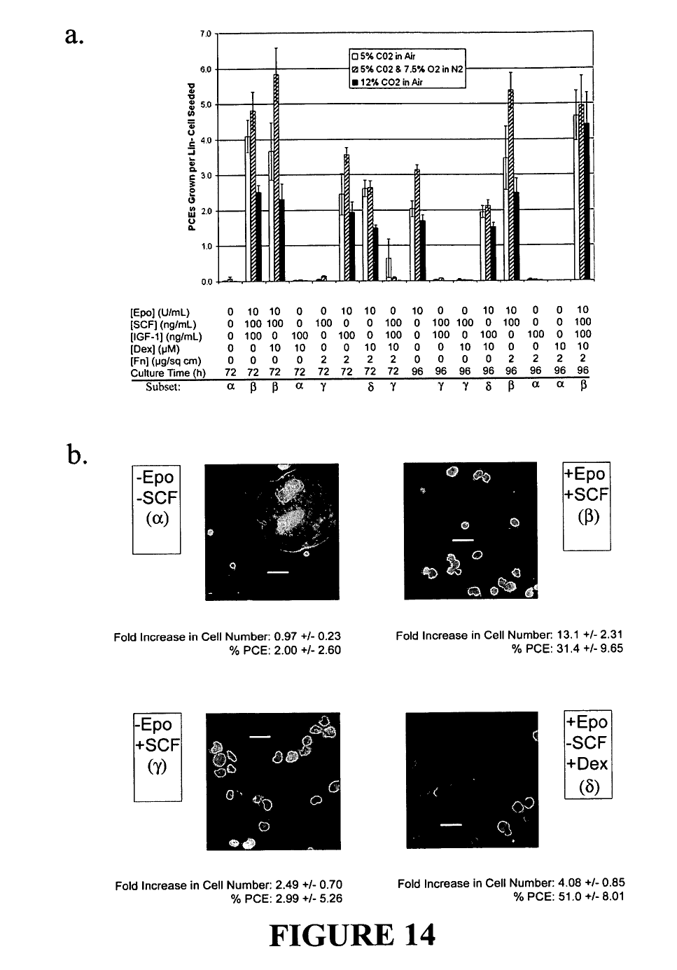

In one embodiment, the culture medium is Iscove's Modified Dulbecco's Media (IMDM) supplemented with defined cytokines, including erythropoietin (Epo) and holo-transferrin, and with fetal bovine serum (FBS). One particularly potent media formulation for inducing erythropoietic growth is IMDM containing 15% FBS, 1% detoxified bovine serum albumin (BSA), 200 μg/mL holo-transferrin, 10 μg/mL recombinant human insulin, 2 mM L-glutamine, 0.1 mM β-mercaptoethanol, 10 U/mL erythropoietin, 100 ng/mL stem cell factor (SCF), and 10 μM Dexamethasone (Dex) (see Table I and FIGS. 11 and 14). In one embodiment, the culture medium is a minimal culture medium. One embodiment of the invention provides an erythroid differentiation culture medium comprising Iscove's Modified Dulbecco Medium (IMDM), 20% fetal bovine serum, 2 mM glutamine, and 0.1 mM β-mercaptoethanol. In one embodiment, the initial culture medium comprises Iscove's Modified Dulbecco Medium (IMDM), 15% (fetal bovine) serum, 1% detoxified bovine serum albumin (BSA), 200 μg/ml holo-transferrin, 10 μg/ml insulin, 2 mM glutamine, 0.1 mm β-mercaptoethanol, 10 U/ml erythropoietin, 100 ng/ml stem cell factor (SCF), and 10 μM dexamethasone.

One embodiment of the invention provides culturing the starting population of cells on a surface comprising fibronectin. For example, the surface can comprise 2 μg/cm² fibronectin.

One embodiment of the invention provides culturing the cells under hypoxic conditions. For example, the hypoxic conditions can comprise 3-15%, e.g., 5-10% 0₂, 3-10%, e.g., about 5% C0₂, and balance N₂.

It may be desirable at times to coculture any of these erythroid progenitor populations with one or several other cell types in an effort to mimic physiological metabolism, transport, and/or cellular interactions. For example, coculture with hepatocytes can be used, as many therapeutic compounds are metabolized by the liver parenchyma. The cells of the present invention can be grown on any surface or vessel which allows erythropoietic growth, including but not limited to liquid culture, semi-solid matrix, stirred-tanks, and perfused bioreactors.

Erythropoietic Culture of Human Cells

The present invention provides a system for the preclinical screening of test compounds on human tissue rather than in animal models. Any culture conditions which support erythropoietic growth of human cells can be used. For example, liquid erythropoietic culture of human PB-MNCs in α minimum-essential medium (aMEM) supplemented with FBS, Epo, and conditioned medium from cultures of the 5637 bladder-carcinoma cell line induces erythropoietic growth, although terminal division and enucleation were only rarely observed using these growth conditions (Fibach, Manor et al. 1989; Fibach and Rα chmilewitz 1993). Specifically, these authors use a two-step liquid culture, with a first phase that is Epo-independent. The media used in the phase I culture, which is 5-7 days in length, is αMEM supplemented with 10% conditioned media from cultures of 5637 cells and 10% FBS and during this phase BFU-Es differentiate into CFU-Es. In the second phase, aMEM containing 1 U/mL Epo, 1 μM Dex, 30% FBS, 1% BSA, and 10 μM β-mercaptoethanol is used to induce the proliferation and further maturation of CFU-Es. After 4 days of phase II culture, centrifugation at 1000 g (for 20 minutes in Percoll with ρ=1.0585 g/mL) is used to effect the removal of lymphocytes from the population, yielding a supernatant that contains the proerythroblasts. These proerythroblasts are then returned to phase II culture for another 10-12 days.

In addition, a three-step culture system can be used to produce fully mature human RBCs from CD34⁺ cells collected from normal BM, PB mobilized with G-CSF, and umbilical cord blood (Giarratana, Kobari et al. 2005). CD34⁺ cells can be cultured in a serum-free base medium supplemented with 1% BSA, 120 μg/mL holo-transferrin, 900 ng/mL ferrous sulfate, 90 ng/mL ferric nitrate, and 10 μg/mL insulin. In the first step (8 days in duration, split after 4 days), these cells are cultured in the presence of 10 μM hydrocortisone, 100 ng/mL SCF, 5 ng/mL interleukin-3 (IL-3) and 3 IU/mL Epo. In the second step, the cells are cocultured with an adherent stromal cell line and stimulated with additional Epo for three days. In the third step, all exogenous factors are withdrawn and cells are incubated on a simple stroma for up to 10 days. In the second and third steps, the stromal feeder layers consisted of either the MS-5 murine cell line or mesenchymal stromal derived from whole normal adult BM. Overall, these authors report an expansion of CD34⁺ HSCs of over 10⁶-fold and a conversion to mature RBC near 100%.

Finally, erythropoietic growth, in culture, from umbilical cord blood has been demonstrated. Light-density MNCs can be isolated from umbilical cord blood samples using Ficoll-Paque density-gradient centrifugation, and then incubated at 37° C. for two hours (Sakatoku and Inoue 1997). Then, the nonadherent MNC fraction can be removed and red cells lysed in 0.75M NH₄Cl. Culture of the resulting population in IMDM supplemented with 20% FBS, 1 U/mL Epo, and 10 ng/mL SCF results in late-stage erythroid development as measured using flow cytometric indicators such as the disappearance of CD34, CD41, and HLA-DR and the appearance of CD71, CD36, and Gly A (Okumura, Tsuji et al. 1992). The representative culture methodologies mentioned above are provided only as exemplary protocols for the induction of terminal erythropoiesis from primary human tissue. As stated previously, any stimulatory ex vivo culture is adequate for the use in the present invention if it induces a signal state, within erythroid progenitors, that triggers terminal division and enucleation.

Test Compounds

The methods of the invention can be used to determine the genotoxic effect of any substance to which a human may be exposed. Test compounds are sometimes referred to herein as test substances or simply compounds or substances. Test compounds include but are not limited to pharmaceuticals, diagnostics, pesticides, cosmetics, vaccines, lotions, foods and packing materials. In one embodiment, the test compound is a therapeutic compound or a diagnostic compound.

In another embodiment, the test compound is a candidate compound for use in treating an erythropoietic developmental defect, including sickle cell anemia, thalassemias, polycythemia vera, and other myeloproliferative disorders. Thus, the methods of the invention can be used to assess both genotoxicity of a compound as well as efficacy in correcting the disease phenotype. For example, in polycythemia vera, too many red cells are produced and the body has lost it's ability to control their production. In this embodiment, the test compound is a candidate compound for use in treating an erythropoietic developmental defect, and the number of MN-PCEs and the number, size, and shape of PCEs are compared between healthy erythropoietic cultures and erythropoietic cultures exhibiting an erythropoietic developmental disorder, in both the presence and absence of the test compound. The test compound can be added to the cells at a concentration which is not cytotoxic to the cells. Genotoxic effects which can be determined using the present invention include clastogenetic effects and aneugenetic effects.

In one embodiment, the test compound is metabolically activated before it is added to the starting population of cells, including by incubation with liver microsomes or a hepatocyte culture.

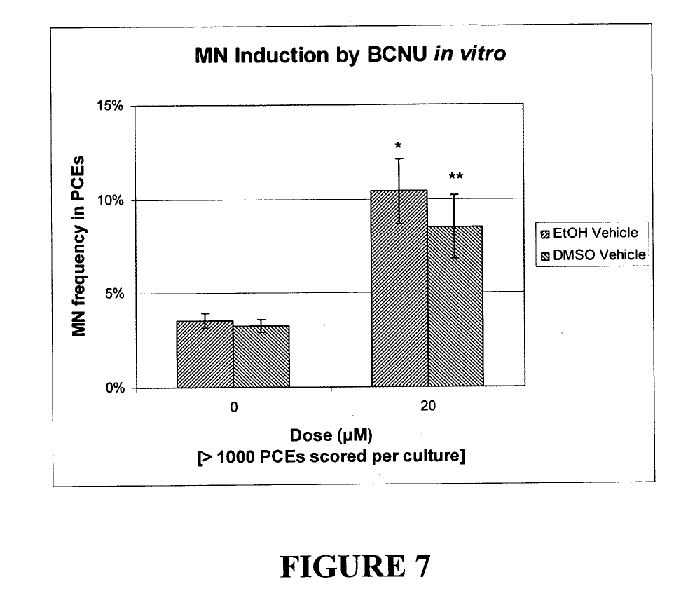

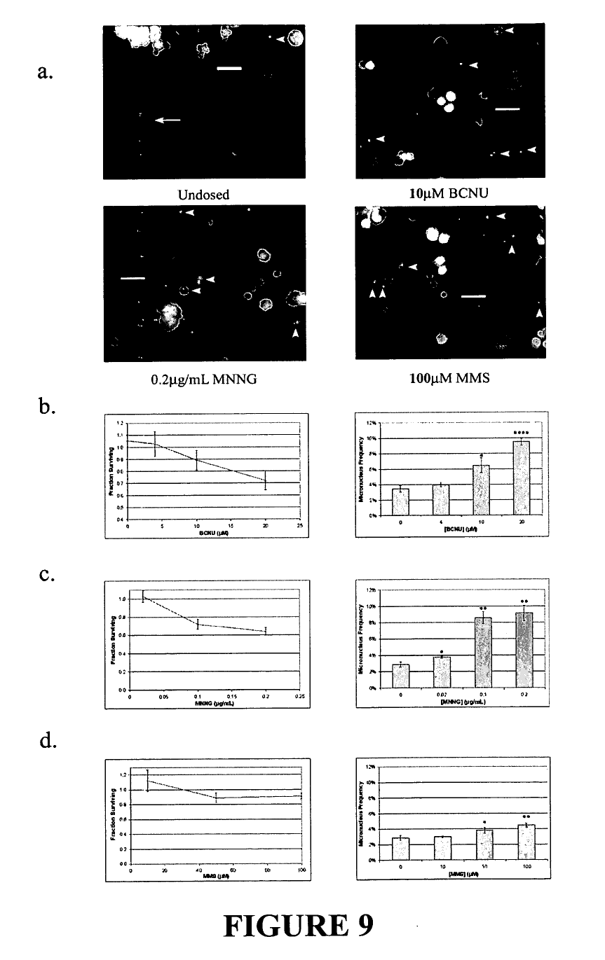

The test compound can be introduced to the erythropoietic culture at an appropriate time and dosage. These times and concentration will differ depending on the chemical and physical properties of the test compound. In one embodiment, the test compound is administered after the erythropoietic population has recovered from isolation and purification stresses but before the erythropoietic population has progressed beyond an early stage of terminal erythropoiesis. Studies with 1,3-bis (2-chloroethyl) -1-nitrosourea (BCNU), a known genotoxicant, indicate that dose delivery after 7 hours of culture at a concentration of 20 μM provides a clear signal of genotoxicity as measured by MN-PCE frequency (see FIG. 7). It can be useful to add the test compound to the cells while in the initial culture medium. When doing so, it may be useful to add the test compound to the cells at a latter stage of culture in this medium, e.g. at hour 23 of culture. Whether the test compound should be washed away following appropriate exposure can also be determined for each specific test compound. Other studies indicate that exposure of test compound after 23 hours of culture also provides useful results regarding genotoxicity (FIG. 9). In one embodiment, the test compound is added to the cells in initial culture medium at 23 hours of culture, and at hour 24, the medium and test compound is washed way, followed by replacement with differentiation culture medium. The amount of time the cells are exposed to the test compound can be optimized for the assay. Results of experiments detailed in the Exemplification section below indicate that exposure of cells to known genotoxicants for as little as 1 hour produce useful measurements of genotoxicity (see FIG. 9 and related discussion). Shorter or longer times of exposure may also be useful.

If metabolic activation of the test compound may alter its genotoxicity, the compound can first be incubated with liver microsomes, or briefly introduced into a hepatocyte culture system, prior to delivery to the erythropoietic culture. For the present invention to provide optimal sensitivity to genotoxicity, the dose of the test agent should not be severely cytotoxic to the erythropoietic culture. This provision ensures the survival of the clonally differentiating progenitors that will ultimately produce a metric of genotoxicity in their PCE progeny.

If a test compound has clastogenic or aneugenic activity, this activity will be reflected by an increase in MN-PCE frequency within the final population. The exact methods used to expose the erythropoietic population to the test compound will vary based on the compound's properties. For example, a small reactive molecule, once dissolved in a benign vehicle, can be added directly to the culture environment and later removed with a media change, if necessary. However, candidate biomaterials, complex metabolic substrates, and biodegradable materials may require additional processing to test the genotoxic potential of their many derivatives.

To maximize sensitivity to genotoxicity, the test compound can be administered at a level that is only slightly toxic to the erythropoietic culture. Inducing a severe toxic effect in the culture system can block the production of PCEs and thus obscure detection of genotoxicity. Furthermore, the test compound can be introduced into culture early enough such that it may act on erythroid progenitors while they are still undergoing differentiation divisions.

To determine appropriate dose levels, one can first test the general cytotoxic effect of a substance in order to ensure survival of some differentiating erythroid progenitors. For example, bone marrow killing curves measured following in vitro dosing with BCNU, a model genotoxicant, indicate that dose concentrations up to 20 μM allow nearly 100% cell survival as measured by colony assay (Glassner, Weeda et al. 1999). Another method for determining appropriate dose levels relies on pharmacokinetic (PK) data collected after in vivo dose administration. The in vivo MN assay introduces test agents into rodents, usually by intraperiotoneal (IP) injection, at doses measured in mass of test agent per mass of animal. It has been determined that, following a bolus injection of 25 mg/kg BCNU, peak plasma concentrations approach 0.4 mM after 1 minute and that these levels decrease to approximately 40 μM within the next 15 minutes (Meulemans, Giroux et al. 1989).

MN and PCE Assays

Any method which detects micronuclei can be used in the methods of the invention. For example, the percentage of cells comprising micronuclei can be determined by flow cytometry, histological analysis and scoring, and automated image analysis platforms.

The culture can be harvested and analyzed after a period of sufficient length to ensure that an adequate portion of the population has completed terminal erythropoiesis. For example, 72 hours is sufficient culture duration for Lin⁻ BM from adult mice. To harvest cells, the culture can be agitated to detach adherent cells and to create a homogenous suspension. Brief incubation with cell dissociation media (e.g., phosphate buffered saline with 5 mM ethylenediamino tetraacetic acid and 10% FBS) can be used to detach any remaining adherent cells. In certain embodiments, harvested samples can be cytospun onto a microscope slide prior to staining and MN-PCE visualization. Alternatively, cell samples can be fixed and stained in situ before using robotic microscopy and image analysis to quantify MN-PCE frequency. A variety of instrument and software platforms offered by Cellomics, Inc. or other companies are capable of performing this type of automated analysis.

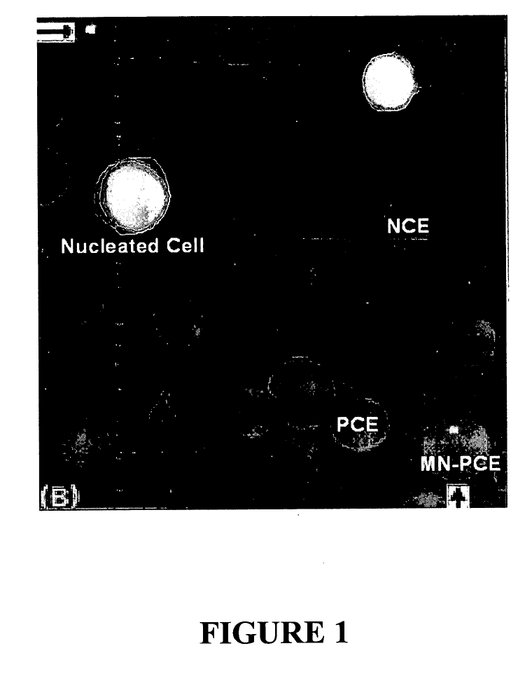

Any visualization technique which allows the detection of MN can be used. In one embodiment, cell samples are air-dried, fixed, and stained with acridine orange (AO) for micronucleus visualization and scoring (Hayashi, Sofimi et al. 1983; Tinwell and Ashby 1989). The application of AO fluorescent staining in the MN test allows the scorer to clearly distinguish DNA from other debris (Hayashi, Sofuni et al. 1983; Tinwell and Ashby 1989). PCEs can be definitively identified using AO staining because they contain single-stranded nucleic acid (RNA) which stains bright orange. NCEs have already translated or degraded all RNA and stain a dull khaki/green color. Finally, AO stains double-stranded nucleic acid (DNA), which is found in nuclei or MN, a bright green (see FIG. 1).

The population sample can be visualized using fluorescent microscopy and scored to determine the percent of newly-formed RBCs that contain micronuclei. In addition to manual visualization and scoring, many methods are available for high-throughput screening of resulting population. Methods include flow cytometry, laser scanning cytometry, and other technologies that incorporate image analysis software to score the resulting samples (Romagna and Staniforth 1989; Dertinger, Torous et al. 1996; Dertinger, Torous et al. 1997; Styles, Clark et al. 2001). Finally, the slides are scored to determine the frequency of micronucleated polychromatic erythrocytes (MN-PCEs) within the PCE population, which will have been largely formed after exposure to the test compound.

Newly formed erythrocytes, sometimes referred to herein as polychromatic erythrocytes or PCEs, contain ribosomes, mitochondria, and mRNA. Mature erythrocytes are sometimes referred to herein as normochromatic erythrocytes or NCEs. PCEs develop into mature erythrocytes over the three to five days, during which the mRNA is translated and/or degraded. PCEs and mature erythrocytes can be distinguished by staining with Acridine Orange. In addition, the ribosomes and mitochondria in PCEs give them a bluish tint after May-Grunwald staining.

Automated methods of population analysis have been developed to provide two main improvements: 1) higher-throughput analysis, and 2) elimination of scorer-subjectivity from the test (Ashby and Mohammed 1986). Flow cytometric analysis based on erythrocyte markers and DNA stains is one approach used for automated scoring (Dertinger, Torous et al. 1996; Dertinger, Torous et al. 1997).

Another approach employs computerized image analysis to score MN-PCEs on slides by searching for regions with low integral, but high peak, DNA-fluorescence intensity (Romagna and Staniforth 1989; Styles, Clark et al. 2001). Standard laser-scanning cytometry (LSC) focuses on a single plane on the surface of the microscope slide and thus does not provide high-quality images of each individual cell. Another alternative is provided by Cellomics® image analysis platforms, which provide cell-by-cell focusing and image data for cells that have been stained in multiwell plates. Thus, a single mouse can provide erythropoietic cultures in, for example, ˜15, 96-well plates, and each well can be treated separately. The cells can be fixed and stained in place, in the plates, before being analyzed by robotic microscopy and image analysis.

Applications

The present invention provides methods for using this culture system, along with automated analysis, to conduct a variety of high-throughput assays, including screening multiple dose-compound combinations for micronucleus induction effects, or for detecting the presence of genotoxic biological and/or chemical agents using the culture system, or for identifying cellular variables that can abrogate micronucleus formation in a given environment.

The invention also provides methods for screening a group of test compounds to determine the genotoxic effect of each individual test compound on erythroid cells, by selecting at least four individual test compounds to comprise the group of test compounds; and determining the genotoxic effect of each individual test compound on an erythroid cell using the methods of the invention. These high throughput methods include screening a group of test compounds simultaneously in a series of parallel cultures. In one embodiment, the group of test compounds comprises at least 30 different individual test compounds. In another embodiment, the group of test compounds comprises at least 300 different individual test compounds. In yet another embodiment, the group of test compounds comprises at least 3000 different individual test compounds. In one embodiment, the genotoxic effect of a test compound is determined at multiple concentrations for that compound, including for example at least 5 different concentrations, or at least 25 different concentrations.

The assay of the present invention is useful for high-throughput screening of test compounds. Optimization of culture conditions, as discussed below, produces sufficient erythropoietic growth to support an assay system for testing hundreds, even thousands of test conditions from a single donor. This in vitro system provides sufficient growth for high-throughput testing from a single mouse. Results of experiments and analyses detailed in the Examples section below indicate that approximately 1850 in vitro erythroid MN assays can be conducted using the hind-leg BM of a single mouse, without further optimization (see FIG. 14a and related discussion).

One application of the present invention, when employed animal tissue, is as an analysis method that serves as a prescreen to be completed before conducting in vivo tests in groups of animals. Furthermore, the invention described here can be practiced using human hematopoietic tissue to better predict clinical responses.

One can use this system to reflect differing in vivo responses to a compound resulting from different genotypes. Thus, hematopoietic cells which are obtained from a donor of a specific genotype will best reflect the genotoxicity on the cells of the particular donor or donor type. This is particularly helpful in better understanding the way different genetic patterns effect drug effectiveness and/or toxicity.

Preclinical toxicity screens typically rely on biological systems that differ from human physiology to some degree. It is believed that the present method more accurately mimics physiologic conditions and can measure the effect of multiple genotoxic agents. This method can be used to study cells such as bone marrow cells in a high throughput and doses specific manner.

Another application of the present invention provides a method to predict the most effective chemotherapeutic regimen for a particular disease state. MN-induction, in vitro, can be used as an indicator of the efficacy of a particular chemotherapeutic regimen. In this application, an increase in MN frequency indicates that a favorable DNA damage response has occurred from the perspective of a desired clinical outcome. The first line of treatment for various types of leukemia is chemotherapy alone or in combination with radiation, depending on the specific cancer. Examples of chemotherapeutic drugs that are used for different leukemias and lymphomas include but are not limited to DNA damaging/alkylating agents including cisplatin, chlorambucil, cyclophosphamide, temozolmide, melphalan, doxorubicin, 5-fluorouracil etc [1]. In some patients, the cancer is constitutively resistant to some chemotherapeutic treatments. The resistance to the desired cytotoxic response to these drugs (e.g. thioguanine, cisplatin, doxorubicin, temozolmide, etc) has been attributed to the loss of the mismatch repair capacity of the targeted cancer cells [2-9]. In some examples, the mismatch repair defective tumor becomes hypersensitive to mitomycin C [10].

In some cases, MN-induction is a result of DNA damage by the previously mentioned chemotherapeutic DNA alkylating agents. If a patient is resistant to a given compound, then the frequency of MNs might be reduced when tissue from this patient is cultured using the techniques previously described. The peripheral blood or bone marrow cells obtained from a patient, when employed in the current invention, can indicate, in their MN-frequency, whether a particular chemotherapeutic regimen will be effective.

Yet another application of the present invention provides methods to predict genotoxicity in an altered state of expression of a particular gene. For example, in some disease states, patients have altered expression of genes. Examples include the loss of BRCA1 and/or BRCA2 genes in breast/ovarian cancer, or the loss of p53 in various tumors. A compound, or combination of test compounds, can be prescreened for genotoxicity in hematopoietic populations that have been induced to display relevant gene expression profiles. In one embodiment one uses autologous tissue from a given patient or cohort. In another embodiment, one can control expression levels either by using RNAi techniques to knockdown genes, or by inhibiting the given gene-product, or by ectopically overexpressing genes in erythroid progenitor populations. Such analyses allow the invention to be tailored to a particular patient or epidemiological cohort.

The invention will be further characterized by the following examples which are intended to be exemplary of the invention.

### EXAMPLES

### Example 1

### In Vitro Micronucleus Assay for Genotoxicity

We have now demonstrated that culturing the lineage-marker negative (Lin⁻) fraction of the bone marrow (BM) from a single mouse under controlled conditions provides sufficient erythropoietic growth to test over 1500 conditions for a genotoxic response.

FIG. 1 shows a micrograph of a purified BM sample that has been stained with acridine orange (AO) and visualized using fluorescence microscopy. AO emits near 515 nm (green) when intercalated in double-stranded (ds) nucleic acid (mostly dsDNA) and near 630 nm (red) when intercalated in single-stranded nucleic acid (mostly RNA). Shown in the image are nucleated cells (green/yellow), NCEs (khaki/green because they are mostly devoid of nucleic acid), and PCEs (bright orange/red mostly due to the presence of ribosomes and mRNA). Also shown is a MN-PCE, which is a newly-formed erythrocyte that contains the remnants of prior genetic damage, either clastogenic or aneugenic, and which is “micronulceated” as a consequence of this damage. It should be noted that a high frequency of nucleated cells will obscure the visualization of the PCEs that ultimately provide a metric of genotoxicity; therefore, BM samples are typically purified to enrich their PCE content prior to slide preparation.

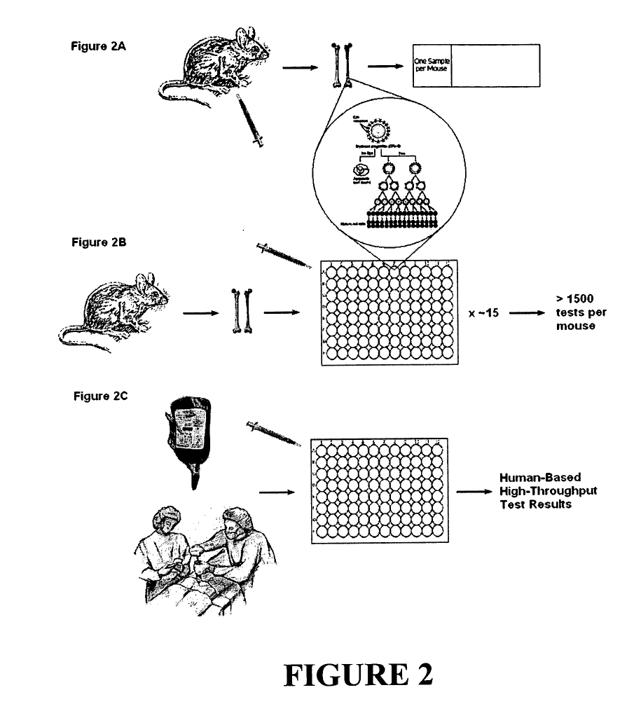

FIG. 2 contains a generalized illustration of the standard in vivo MN assay (FIG. 2A) as well as generalized illustrations of the present invention conducted in vitro using both rodent (FIG. 2B) and human (FIG. 2C) hematopoietic populations. In exemplary FIG. 2A, a test agent is delivered to a mouse; some time later, the mouse is sacrificed and BM from its hind-leg femurs is used to create a unique biological sample. Prior to spreading on a slide, the BM-derived cell suspension would typically be passed through a cellulose column to enrich the PCE content by removing a portion of all nucleated cells, which are retained in the column. Then the biological sample is typically fixed on the slide and stained with AO prior to visualization by fluorescence microscopy, which yields image fields similar to that shown in FIG. 1.

In exemplary FIG. 2B, the mouse is first sacrificed and the BM is removed to provide a primary erythropoietic population; if desired, this population can be enriched for erythroid progenitors and depleted of preexisting PCEs by a variety of separation techniques. The primary erythropoietic population is then suspended in culture media formulated to induce erythropoiesis, and aliquots of the suspension are seeded into the various wells of several 96-well culture plates. Multiple test compound/dose-concentration/dose-time combinations are then delivered to these erythropoietic cultures to create multiple, biologically-distinct samples. Then, these samples can either be removed from the culture environment prior to analysis, or the samples can be fixed, stained, and visualized in situ. Erythropoietic growth and the location where this growth occurs is also illustrated in FIGS. 2A and 2B. Exemplary FIG. 2C illustrates the conduct of the present invention using primary human tissue, in this case using erythroid progenitors in donor PB or donor BM. In FIG. 2C, the methods represented by the various arrows are analogous to those provided for FIG. 2B above.

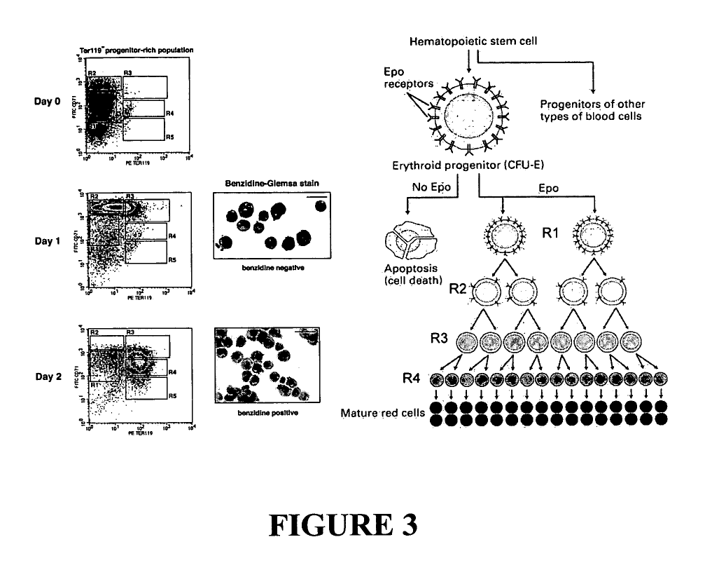

FIG. 3 is an illustration of a flow cytometric technique that can be used to track the progress of erythropoietic growth in culture.

Recent advances in the analysis of late-stage, in vitro erythropoiesis allowed the current inventors to rationally modify culture conditions and thus facilitate the high-throughput genotoxicity testing of multiple conditions using the hematopoietic tissue of a single animal and the metric of MN-PCE frequency. The work of two of the current inventors provides a culture environment for ex vivo erythropoiesis and also establishes a flow cytometric technique to track, with high resolution, the final divisions of erythrocyte differentiation (Socolovsky, Nam et al. 2001; Zhang, Socolovsky et al. 2003). Flow cytometric analysis of erythrocyte differentiation is achieved by double-staining for CD71 and Ter-119. Ter-119 is a molecule that is associated with Gly A, and antibodies against Ter-119 specifically bind the surface of cells in late stages of erythroid differentiation (Kina, Ikuta et al. 2000). Late-stage erythropoietic cells express CD71 briefly during differentiation. Therefore, in FIG. 3, R1 cells represent the most primitive erythroid cells. As R1 cells differentiate, they sequentially enter regions R2, R3, R4 and R5, in that order.

Over the course of late stage erythropoietic culture, the flow cytometric staining characteristics of a Ter-119⁻ murine erythroid progenitor population changes in a predictable manner as its members undergo three to five terminal cell divisions and differentiate into PCEs (see FIG. 3) (Lodish 2003). The ability to track these last developmental divisions by flow cytometry allowed the present inventors to quantify the net production of erythroid cells, at various stages of differentiation, in a dynamic manner and thereby quantify erythropoietic stimulation in several different growth environments. Briefly, total cell counts were conducted upon each population harvest, and then the percentage of this population that fell within a given erythroid flow cytometric regions, as depicted in FIG. 3, was determined. The combination of these two quantities allowed the net production of cells in a given, late-stage of er)throid development to be calculated.

In FIG. 3, the abscissa and ordinate on these density plots both represent log-scale relative fluorescence, and the former provides a measure of Ter-119 expression while the latter provides a measure of CD71 expression. As erythropoietic growth occurs in vitro, a population that is initially devoid of Ter-119, an erythroid-specific cell surface protein, begins to express Ter-119 after first increasing its expression of CD71, the transferrin receptor. In terms of the flow cytometric regions drawn in these plots, the cells progress from stages R1 and R2 into stages R3, R4, and finally R5 as they differentiate into erythrocytes. Here, erythropoietic growth was induced from Ter-119⁻ FL over a culture period of two days.

Also shown in FIG. 3 are histological samples from these cultures at day 1 and day 2 that have been stained with benzidine-giemsa stain; yellow coloration in the day 2 population indicates that the cells are benzidine positive and, consequently, that the cells contain hemoglobin. The scale-bar in these images is 20 μm. Juxtaposed with these flow cytometric and histological images is an artist's rendition of erythropoietic growth, with each stage of differentiation labeled with the flow cytometric region that it approximately corresponds to. These images were taken from published documents(Lodish 2003; Zhang, Socolovsky et al. 2003).

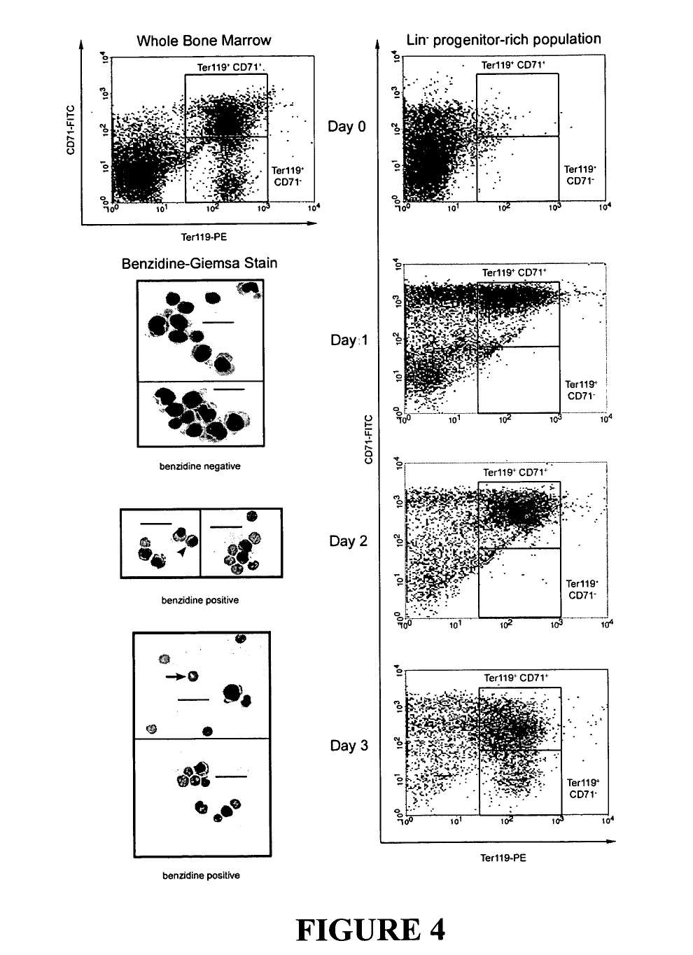

FIG. 4 tracks the terminal erythropoiesis stimulated in Lin⁻ bone marrow cells over 3-days in culture. Bone marrow cells were stained with biotinylated α-lineage marker (α-Lin) mabs (α-CD3e, α-CD11b, α-CD45R/B220, α-Ly6G/Ly6C, and α-TER-119), and the Lin⁺ fraction of the population was subsequently removed to obtain a progenitor-rich population (Lin⁻ bone marrow). The Lin⁻ fraction of mouse BM was isolated by immunomagnetic negative selection. These Lin⁻ cells were then cultured in vitro for 3 days on fibronectin-coated plates in medium containing serum. Epo was included in the medium for the first day of culture, and then the medium was changed and Epo was removed. The seeded Lin⁻ cells and the resulting populations at various stages of culture were analyzed by flow cytometry and benzidine-Giemsa stain to track erythropoietic differentiation in this system (FIG. 4).

Flow cytometry indicated that a significant portion of Lin⁻ cells had begun erythroid differentiation after 1 day of culture with Epo (FIG. 4). However, not all of the Lin⁻ population responds to Epo stimulation, and a portion remains Ter-119⁻/CD71⁻ at the end of the first day in culture (FIG. 4). These unresponsive cells are absent in culture at later time points, and presumably few cells that are Ter-119⁻/CD71⁻ at day 1 survive until day 2. The majority of Lin cells that do survive the culture period undergo the characteristic late-erythroid changes in surface expression that have been reported for both in vivo fetal liver and cultured Ter-119⁻ fetal liver (Socolovsky, M. et al. Blood 98, 3261-3273 (2001); Zhang, J. et al., Blood 102, 3938-3946 (2003)). After the third day of culture, flow cytometry indicated that the majority of the resulting population had acquired a late erythroid surface phenotype (Ter-119⁺). Furthermore, benizidine Giemsa stain revealed that many cells in the harvested population were enucleated and expressing hemoglobin. Benzidine-Giemsa histology further confirmed that a substantial portion of the Lin⁻ BM population (hemoglobin⁻ and nucleated) differentiated into fully mature reticulocytes (hemoglobin⁺and enucleated) over the course of 2 to 3 days in this culture system (FIG. 4).

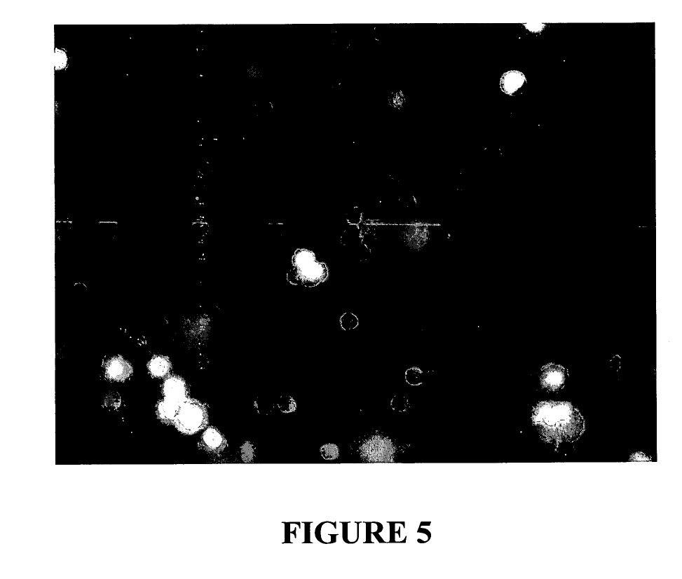

FIG. 5 is a representative image field of a population following erythropoietic culture. Specifically, this image is of Lin⁻ mouse BM from C57BL/6J mice, aged 6-8 weeks, that was cultured under condition #7 (defined in Table I). Following harvest, the cells were fixed and stained with AO, which gives specific fluorescence signals as detailed in the description of FIG. 1. It should be noted that PCEs are the majority cell type following culture under condition #7 and that no purification step, such as that used to generate FIG. 1, is required to easily visualize PCEs following erythropoietic culture. PCEs are also the majority cell type following culture under condition #12 (see Table I), and are present in high percentages, and are thus easily distinguished, following most cultures that include sufficient Epo. Also note the presence of a spontaneous MN-PCE near the center-left of this image. The absence of NCEs indicates that all of these enucleated erythrocytes where formed during the three-day culture period that generated this population, and consequently that the Lin⁻ BM was essentially free of PCEs prior to culture.

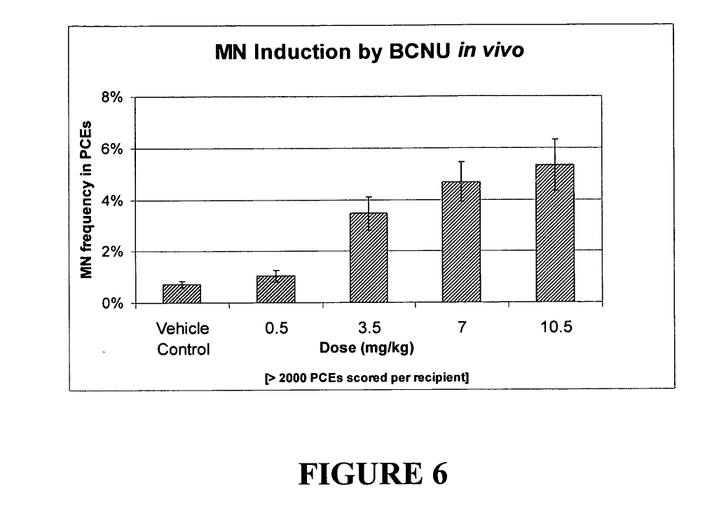

FIG. 6 is a plot of the dose-response to BCNU as measured in C57BL/6J mice by the standard in vivo MN assay. BCNU, a known genotoxicant that has been shown to form interstrand cross-links and that is known to behave as a clastogen, was administered by IP injection and animals were sacrificed 24 hours prior later. Slides were then prepared as described elsewhere in this document and the MN frequency within the PCE population was determined. The MN frequency in the PCE population continues to increase with increasing BCNU dose up to the maximum dose tested (10.5 mg/kg), illustrating the ability of the in vivo MN assay to detect dose-dependent genotoxicity. Here, 24 mice were used to generate the data shown, with 8 mice treated at the 7 mg/kg dosage and 4 mice treated at each of the other doses. Over 2000 PCEs were analyzed per animal; the error bars represent the mean plus or minus one standard deviation.

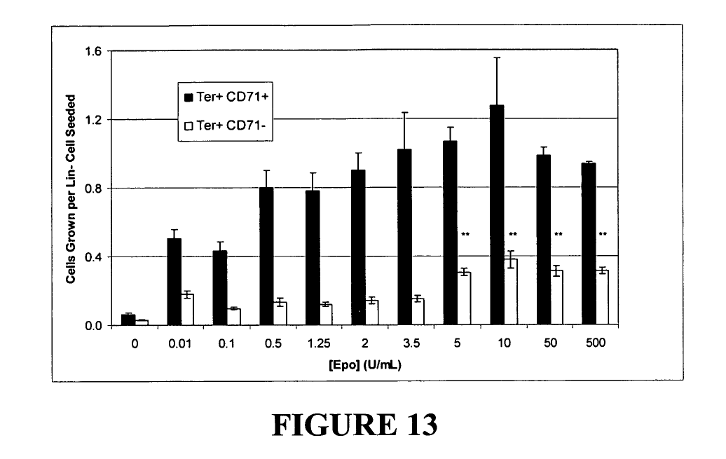

FIG. 7 is a plot of the dose-response of genotoxicity as detected by MN frequency to BCNU as measured by conducting the present invention using the Lin⁻ fraction of C57BL/6J (mouse) BM as an initial population. FIG. 13 demonstrates the ability of the present invention to detect genotoxicity using the metric of MN frequency within the PCE population. BCNU or vehicle control (dimethyl sulfoxide or ethanol) was introduced into erythropoietic culture 7 hours after seeding. After 72 hours in culture, the resulting populations were harvested and slides were prepared as described elsewhere in this document. The MN frequency in each PCE population was then determined by microscopic examination. Three cultures were produced at each dose condition, and the error bars represent the mean of these biological triplicates plus or minus one standard deviation. Here, two mice were used to generate the data shown, with over 1000 PCEs analyzed per culture. The MN frequency in cultures exposed to BCNU at an initial concentration of 20 μM was significantly different than those exposed to vehicle control alone (* indicates P(T≦t) <0.022 and ** indicates P(T≦t) <0.036 as determined by a two-tailed t test).

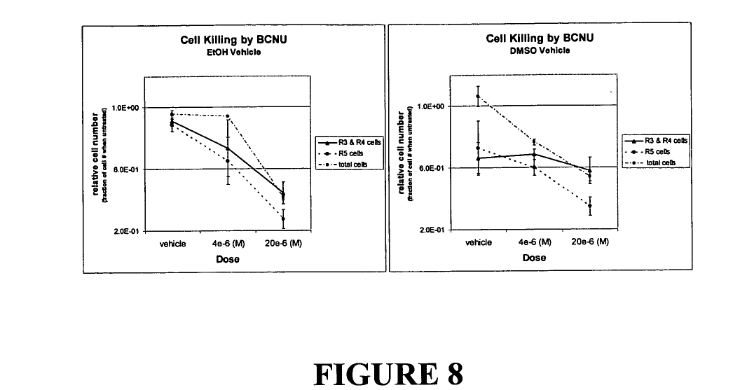

FIG. 8 is a plot of the dose-response to BCNU as measured by counting cell numbers from dosed cultures and then plotting these as relative values of untreated (control culture) cell numbers. The figure provides data using two different vehicles; data for ethanol is presented on the left and data for dimethyl sulfoxide is presented on the right. Cell numbers, relative to those from untreated cultures, are presented both for total cells and for erythroid specific cells. These data were obtained in parallel with the data acquisition for FIG. 7; that is, the vehicle and 20 μM cultures that provided these data were the same cultures as those used to generate FIG. 7. The fractions of the harvested populations that were contained within the “R3 & R4” and “R5” erythropoietic regions (see FIG. 3) were determined by two-color flow cytometry, and these fractions were used to convert total cell numbers to erythroid-specific cell numbers. The data here show that increasing doses of BCNU decrease both total and erythroid specific cell numbers, and that the use of ethanol as a vehicle may contribute to this cytotoxicity. Taken together with FIG. 7, these results indicate that the present invention can be used to detect BCNU-induced cytotoxicity, and that a concomitant increase in MN frequency (see FIG. 7) can provide a clear indication that this observed toxicity is genotoxic in nature.

FIG. 9 provides additional examples of detection of genotoxic exposure through an in vitro erythroid micronucleus assay. Purified Lin⁻ cells were cultured in vitro for 1 day on fibronectin-coated plates in media containing serum, Epo and other erythropoietic growth factors (SCF, Dex, and IGF-I). Three mutagenic alkylating agents that test positive in the in vivo MN assay (1,3-bis[2-chloroethyl]-1-nitrosourea [BCNU], N-methyl-N-nitro-N-nitrosoguanidine [MNNG], and methylmethane sulfonate [MMS]) were selected as model genotoxicants and were introduced into erythropoietic cultures. These genotoxicants were added to erythropoietic cultures at a range of concentrations 23 h after seeding and were then removed by media exchange 1 h later (24 h after seeding). In the course of the media exchange, the media was changed and all soluble growth factors and alkylating agents were removed. Populations were then cultured for 2 additional days in media with serum.

At harvest, cells of the dosed and control populations were removed from culture, viable cells were counted, and samples were stained with acridine orange to visualize PCEs and enumerate MNs within the PCE population (Fig BA taken at 3 d in culture). Genotoxic effects were quantified through total viable cell counts and differential cell counts. The fractional survival of treated cultures was determined by normalizing the mean of viable cell counts from those cultures to the mean of viable cell counts from untreated cultures. The micronucleus frequency in cultures was determined by acridine orange stain and differential cell counts (>2000 PCEs scored per culture).

The effect of these 3 genotoxicants on viable cell numbers indicates that all of these compounds have a slight toxic effect in this system at the doses examined (left-hand panels of FIG. 9b, 9c, and 9d). More importantly, the MN frequency in the PCE population increases with the concentration of these alkylating agents and, at higher doses, the mean MN frequency in treated populations was significantly higher than that in untreated populations (right-hand panels of FIG. 9b, 9c, and 9d). Taken together, these data further demonstrate that genotoxic exposure can be detected using this erythropoietic culture system.

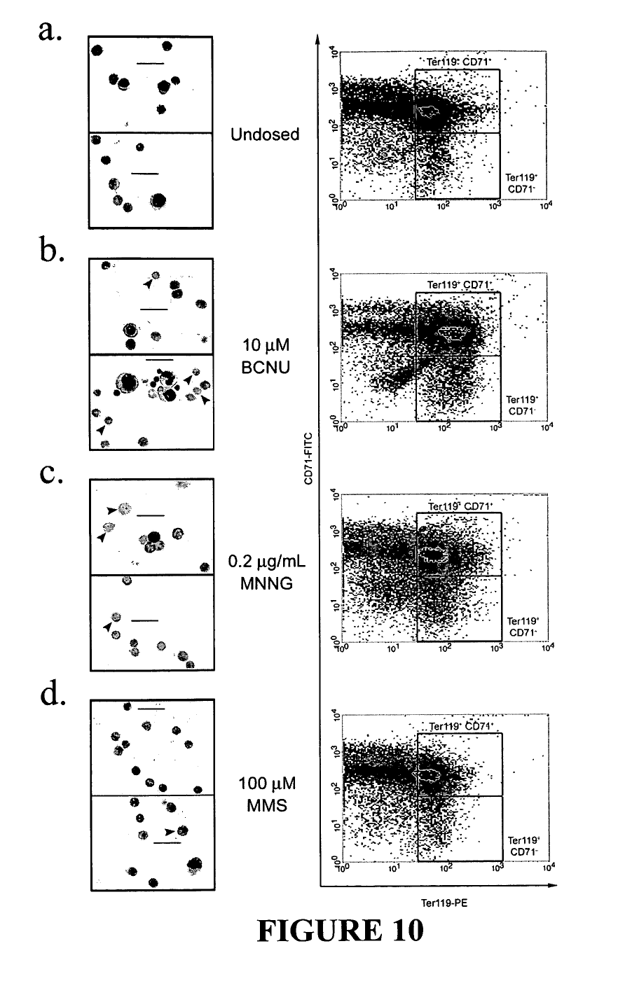

The cultured populations harvested for FIG. 9 were also analyzed by flow cytometry and benzidine-Giemsa stain. The results are shown in FIG. 10. Histology revealed that large fractions of the treated populations were hemoglobin⁺ and fully enucleated. In addition, large fractions of the dosed populations were found by flow cytometry to express the characteristic late-erythroid surface markers Ter-119 and CD71. This indicated that erythropoiesis had progressed normally in these populations despite treatment with genotoxicants (FIG. 10).

### Example 2

### Culture Conditions for Erythroid Growth

The flow cytometric techniques described in Example 1 were originally developed for E14.5 fetal liver, and developing an analogous culture system from adult erythroid progenitors required a modified starting population. While approximately 41 percent of R1 cells in FL are CFU-Es, R1 cells in BM contain the committed progenitors and differentiated progeny of a variety of hematopoietic lineages. Fortunately, a detailed knowledge of the cell-surface markers of murine CFU-Es exists (Terszowski, Waskow et al. 2004).

To develop improved culture technology, the potential of Ter-119⁻ BM and Lin⁻ BM, obtained from C57BL/6J mice, to yield PCEs in culture was examined (see Tables I and II, and FIGS. 11, 12, 14, and 16). Initial studies revealed that either population, when cultured in the presence of Epo for approximately 72 hours, could be induced to undergo some degree of terminal erythropoiesis. However, Lin⁻ mouse BM displayed greater sensitivity to erythropoietic stimulatory factors and was thus used as a model erythropoietic tissue for the development of improved culture methodologies.

TABLE I
Details of Culture Conditions Used in Experiments

| Condition | [Epo] (U/mL) | [SCF] (ng/mL) | [IGF-1] (ng/mL) | [Dexamethasone] (μM) | [Fn] (μg/cm²) | Culture time (h) |
|---|---|---|---|---|---|---|
| 1 | 0 | 0 | 0 | 0 | 0 | 72 |
| 2 | 10 | 100 | 100 | 0 | 0 | 72 |
| 3 | 10 | 100 | 0 | 10 | 0 | 72 |
| 4 | 0 | 0 | 100 | 10 | 0 | 72 |
| 5 | 0 | 100 | 0 | 0 | 2 | 72 |
| 6 | 10 | 0 | 100 | 0 | 2 | 72 |
| 7 | 10 | 0 | 0 | 10 | 2 | 72 |
| 8 | 0 | 100 | 100 | 10 | 2 | 72 |
| 9 | 10 | 0 | 0 | 0 | 0 | 96 |
| 10 | 0 | 100 | 100 | 0 | 0 | 96 |
| 11 | 0 | 100 | 0 | 10 | 0 | 96 |
| 12 | 10 | 0 | 100 | 10 | 0 | 96 |
| 13 | 10 | 100 | 0 | 0 | 2 | 96 |
| 14 | 0 | 0 | 100 | 0 | 2 | 96 |
| 15 | 0 | 0 | 0 | 10 | 2 | 96 |
| 16 | 10 | 100 | 100 | 10 | 2 | 96 |

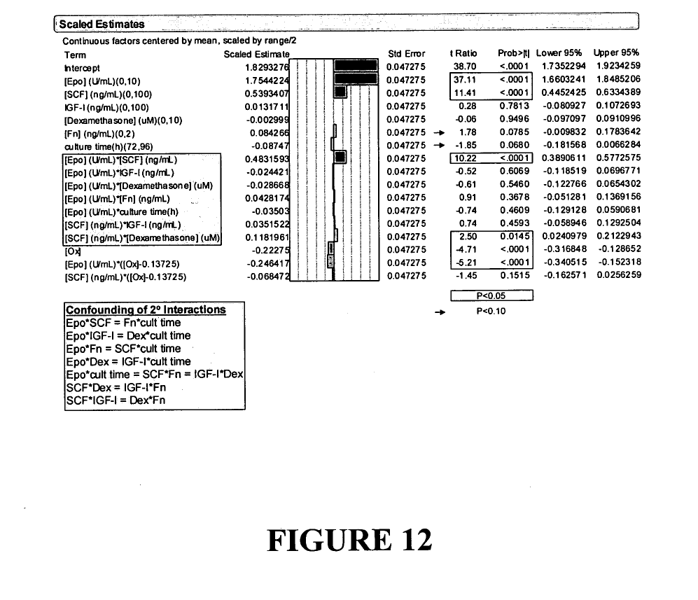

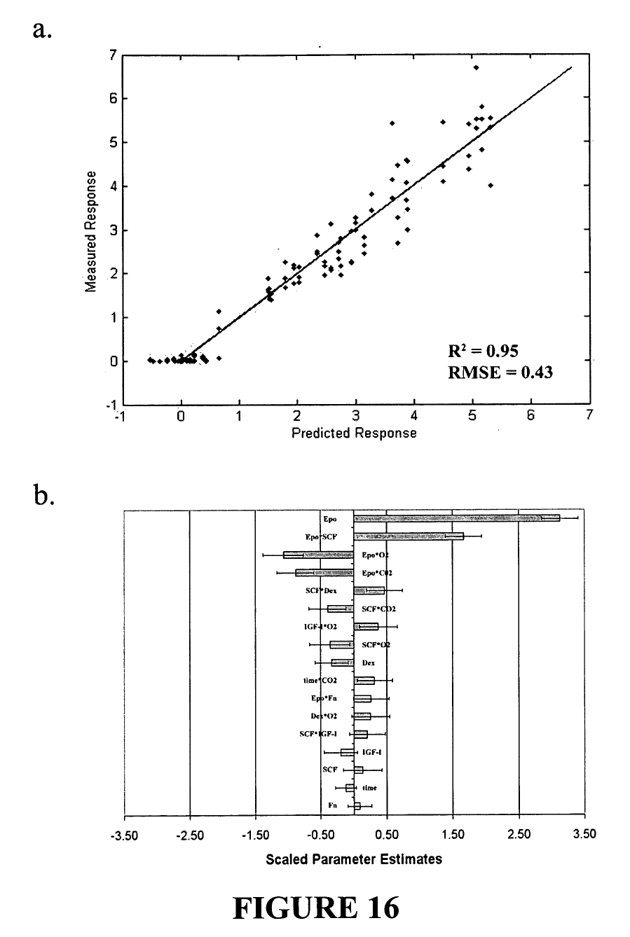

Table I is an experimental design chart that provides the specific details of the experimental conditions that were examined to generate FIGS. 11, 12, 14 and 16. Specifically, this experimental design is a two-level, minimum-aberration, fractional-factorial design of resolution IV (a 2_{IV}⁶⁻² minimum-aberration design). The full-factorial design was not performed because an experiment of this size (2⁶ cultures for each biological singlet) was logisitically infeasible. This experiment was designed in order to estimate all the main (primary) interactions between the culture response (PCEs produced) and the individual parameters included in the design. In addition, many of the secondary interactions (between the culture response and various parameter-pairs) were estimable by conducting this design (see FIGS. 12 and 16), although some of these secondary interactions were confounded with other secondary interactions. Each condition listed in the design table was tested in biological triplicate, and each triplicate set was cultured under three different atmospheric conditions, to yield a total of nine culture wells per listed condition.

FIG. 11 provides results from the experiment for which Table I provides the design. The various atmospheric conditions examined are indicated in the legend, while the numbers (1-16) correspond to the culture conditions detailed in Table I. Upon harvest of each culture, a total cell count was taken and representative slides were prepared and stained to provide estimates of the fraction of the harvested population that consisted of PCEs. Over 2000 cells were visualized and counted per slide in order to estimate the PCE fraction in the total population. Based on the results from culture under condition #3 (in reduced oxygen), which is the most promising condition tested, 5.8±0.8 PCEs can be produced per input Lin⁻ mouse BM cell. Here, C57BL/6J males aged 6-8 weeks were used as a source of primary tissue. Over 5×10⁵ Lin⁻ BM cells can be obtained from the hind-leg femurs and tibias of these mice. Using these numbers, it is estimated that more than 1600 distinct PCE populations, each containing 2000 members, can be generated using the hind-leg BM from a single adult mouse. The data set from culture under the third atmospheric condition (12% CO₂, balance Air) does not appear in this figure, but appears in FIG. 14.

FIG. 12 provides the results from numerical modeling conducted to estimate the primary and secondary effects of the independent parameters that were studied in the experiment that is described by FIG. 11. It was found that Epo, Oxygen, and SCF were the major factors influencing erythropoietic growth of Lin⁻ BM from C57BL/6J mice and that these effects were statistically significant (P(T≦t)<0.001). Fn was also found to have a significant, primary influence on PCE production (P(T≦t)<0.05). In addition to the main (primary) contribution of Oxygen and SCF these factors also displayed statistically-significant secondary interactions with Epo, and a positive secondary interaction was also observed between SCF and Dexamethasone. Finally, these data suggest that a harvest time near 72 hours is preferred to a harvest time near 96 hours. It should also be noted that these results were obtained from untreated cultures. Treatment with a toxic test agent may alter culture responses. Note, this analysis was performed in two different manners, the first manner being presented and discussed here, and the results of the second manner presented in FIG. 16 and discussed below.

Results

Studies were conducted by stimulating Lin⁻ mouse BM with specifically defined media formulations for 24 hours before replacing the medium to a minimal erythroid-differentiation medium (EDM) and culturing for an additional 2-3 days. This EDM contains 20% FBS, 2 mM L-glutamine, and 0.1 mM β-mercaptoethanol in IMDM, whereas the “Day 1” medium consists of IMDM with 15% FBS, 2 mM L-glutamine, 0.1 mM β-mercaptoethanol, 1% BSA, 200 μg holo-transferrin, 10 μg/mL insulin as well as further supplements (some specific media formulations listed in Table I). Upon harvest, the number of late-stage erythroid cells and enucleated erythrocytes produced over the culture period was analyzed. Flow cytometric methods, as described above, were used in initial effect screening studies to identify culture factors that had a pro-erthyropoietic effect and to gauge the relative size of these effects. Although the most relevant endpoint for these cultures is PCEs-produced per primary BM cell, the number of R4 and R5 cells in the post-culture population, as measured by flow ctyometry, provided a reasonable metric of PCE content for early screening studies. More rigorous quantification, via histological analysis, was then employed in a more detailed analysis to generate FIGS. 11, 12, 14 and 16.

One supplement added to the basic “Day 1” media formulation is Epo, which is an essential component of the erythropoietic media formulation. Epo promotes the survival of colony-forming units erythrocyte (CFU-Es), thus facilitating their differentiation into reticulocytes (Lodish 2003). Flow cytometric analyses conducted on cultured Lin⁻ BM found that the erythropoietic stimulatory effect of Epo reached a maximum at a level of approximately 10 U/mL. There is also prior evidence that SCF has a stimulatory effect on erythropoietic growth, and the SCF receptor, c-Kit, has even been identified as a marker for the isolation of CFU-Es (Socolovsky, Fallon et al. 1998; Terszowski, Waskow et al. 2004; Waskow, Terszowski et al. 2004). Again, flow cytometric analyses, using the methods and experimental system described above, revealed that SCF did induce increasing erythropoietic growth up to the maximum concentration tested, which was 100 ng/mL (Table II). Furthermore, coating the culture surface with Fn promotes stimulatory adhesion of CFU-Es before they differentiate and decrease expression of the Fn receptor (Patel and Lodish 1986). In the case of Fn, erythropoietic flow cytometry revealed a small effect when coated plates were compared with uncoated plates, but this effect could not be enhanced with increasing Fn concentration. That is, a coating of 2 μg/cm² was found to provide approximately all of Fn's stimulatory effect (Table II).

Another stimulatory factor that is sometimes added to erythropoietic liquid culture is Dexamethasone, also referred to as Dex. (Fibach and Rα chmilewitz 1993). Dex has been found to have erythropoietic activity in vivo, and it is known that Dex stimulates the glucocorticoid receptor to induce a cooperative erythropoietic response stemming from simultaneous stimulation with Dex, Epo, and SCF (Malgor, Barrios et al. 1987; von Lindem, Zauner et al. 1999). Furthermore, IGF-I has been found to have an erythropoietic stimulatory effect both in vivo and in vitro, and an in vitro study by Sawada and Krantz suggests that IGF-I stimulates erythroid progenitors directly, rather than through the action of accessory cells (Sawada, Krantz et al. 1989; Bechensteen, Halvorsen et al. 1994). In addition, a multifactor analysis found that the stimulatory effects of SCF, Epo, Dex and IGF-I could be employed, in concert, to provide an increasingly proliferative erythropoietic environment{Panzenbock, 1998 #607). Therefore, the flow cytometric methods were used to quantify the erythropoietic effect of Dex and IGF-I on Lin⁻ BM from C57BL/6J mice. These preliminary studies found that these factors also had an effect on erythroid cell numbers in the present experimental system (Table II).

In addition, it is known that basic chemical properties, such as media pH and dissolved oxygen content, which is relative to atmospheric oxygen content, can have an effect on erythropoietic growth. Normally, pH is controlled by the addition of NaOH or HCl to culture media, and it has been found that a pH near 7.6 induces greater erythropoietic growth than a pH of 7.35 or 7.1 (McAdams, Miller et al. 1998). However, the present inventors found that the % CO₂ in the incubation atmosphere has a dominant effect on pH over extended culture, and can be used to control pH in a more direct and robust manner. Experiments conducted at pH 7.6 (2.5% CO₂) and pH 7.4 (5% CO₂) revealed a contrary result in the present experimental system; that is, more erythropoietic growth was observed at the lower pH as measured by erythropoietic flow cytometry. Regarding atmospheric O₂, we have confirmed that a hypoxic ambient condition in the incubator (5-10% O₂, 5% CO₂, balance N₂) can enhance erythropoietic growth, as measured by erythropoietic flow cytometry in the present experimental system. This finding regarding hypoxic culture is consistent with previous research reports in the literature (Ishikawa and Ito 1988; Koller, Bender et al. 1992; Laluppa, Papoutsakis et al. 1998). Finally, the duration of culture was found to have an effect on erythropoietic growth, with a maximum fraction of the population reaching the R4 and R5 stages of erythropoietic growth between after 72-96 hours in culture.

However, one study that compared erythropoietic flow cytometry region statistics with PCE fractions, as determined by histological examination of population samples, revealed that flow cytometry does not provide a rigorous metric of PCE production. Furthermore, some variability between various Lin⁻ BM isolations was observed over the course of preliminary flow cytometric studies. Therefore, a definitive study was conducted that used aliquots of a large, well-mixed Lin⁻ population that was isolated from the combined BM of 15 mice. In this study, flow cytometry was replaced by histological analysis to provide more rigorous quantification of a population's PCE content. This study was designed to estimate the relative stimulatory effect of various, previously-identified erythroid growth factors on culture performance. Specifically a minimum-aberration, fractional-factorial experimental design (a 2_{IV}⁶⁻² minimum-aberration design) was conducted to estimate all the main (primary) interactions between the culture response, which was PCEs produced (as assessed by histology), and the individual parameters included in the design (see Table I, and FIGS. 11, 12, 14 and 16). In addition, many of the secondary interactions (between the culture response and various parameter-pairs) were estimable by conducting this design, although some of these secondary interactions were confounded with other secondary interactions.

Each culture condition listed in Table I was performed in biological triplicate. Furthermore, each of these experimental sets of 48 cultures (16 conditions in triplicate) was simultaneously conducted in three different atmospheres. The first of these was a standard atmosphere of 5% CO₂ in air; the second was a hypoxic atmosphere of 7.5% O₂ and 5% CO₂ (balance N₂); and the third was an atmosphere of 12% CO₂ in air that had been found to lower the media pH to approximately 7.1. The results of total cell counts, combined with histological data to give total PCEs produced, is presented in FIG. 11. It should be noted that hypoxic condition #3 (see Table I) provides the highest overall yield of PCEs, and that conditions #7 and #12 result in the growth of populations that contain greater than 50% PCEs (see FIG. 5).

Statistical analysis was conducted to gauge the relative effect of the various parameters included in this erythropoietic optimization study. FIG. 12 displays the scaled estimates of all the primary and secondary effects of the chosen culture parameters. By this analysis it was found that the largest, and most statistically-significant, effect was provided by Epo, with SCF and a synergistic SCF-Epo interaction providing the next largest effects. Furthermore, decreased oxygen had a significant direct stimulatory effect on erythropoietic culture, and a synergistic ox-Epo secondary effect was also observed. In addition, Fn provided a slight stimulatory effect on erythropoietic growth in this experimental system. Dex alone did not have a statistically significant effect on the production of PCEs, but it should be noted that conditions #7 and #12, which produced highly pure PCE populations, both contained Dex without SCF. In certain applications of the present invention, a culture that produces pure erythropoietic populations may be preferred to one that induces growth of large and impure populations. Finally, it was found that a culture of 72 hours was sufficient to obtain terminal differentiation and that no positive effect on PCE production is provided by extending the culture to 96 hours. Based on the levels of PCE production provided by hypoxic condition #3, the present invention can be used to produce over 1500, biologically-distinct populations that each contain 2000 PCEs.

Based on the in vitro survival and in vivo PK data described above, a physiologically-relevant BCNU test concentration of 20 μM was selected and introduced into the erythropoietic culture system 7 hours after culture inoculation. The results of this experiment show that this BCNU dosage reduces both total and erythroid cell numbers, indicating a cytotoxic response in erythropoietic Lin⁻ BM (see FIGS. 8 and 9). Most importantly, the clastogenic nature of this toxicity was clearly indicated by an increase in the frequency of MN-PCEs (see FIG. 7 and 9). Thus, this in vitro culture system can be used to detect cytotoxicity while also providing a clear signal when the observed cytotoxicity is caused by a genotoxic mechanism (clastogenicity or aneugenicity).

As shown above, short-term erythropoietic culture can be used to detect genotoxic exposure. Quantitative studies on the erythropoietic growth of Lin⁻ BM after stimulus with physiologic erythropoietic factors were used to further improve the culture methodology and thus increase the potential throughput of the in vitro erythroid MN assay described here. Initially, flow cytometry was used to define late-erythroid cells by subdividing the Ter-119⁺ population based on CD71 expression (regions shown in FIG. 4) to estimate the fraction of a culture that acquired late-erythroid surface markers. The products of flow cytometric region statistics and total cell counts were calculated and then normalized to input cell numbers to quantify erythropoietic growth in a culture.

In one such study, it was found that the stimulatory erythropoietic effect of Epo on Lin⁻ BM reached a maximum at approximately 10 U/mL (FIG. 13). A significant stimulatory effect was also observed for several other physiologic erythropoietic stimuli (Table II), and many of these factors were then incorporated into a 2-level, orthogonal, fractional-factorial factorial experiment to estimate primary and secondary parameter effects (experimental design represented on the abscissa of FIG. 14A) (Bechensteen, A. G., et al., Acta Physiol Scand 151, 117-123 (1994); Ishikawa, Y. et al., Eur J Haematol 40, 126-129 (1988); Koller, M. R., et al., Exp Hematol 20, 264-270 (1992); LaIuppa, J. A., et al., Exp Hematol 26, 835-843 (1998); Lodish, H. F. Molecular cell biology, Edn. 5th. (W. H. Freeman and Company, New York; 2003); Malgor, L. A. et al. Acta Physiol Pharmacol Latinoam 37, 365-376 (1987); McAdams, T. A., et al, Br J Haematol 103, 317-325 (1998); Panzenbock, B., et al., Blood 92, 3658-3668 (1998); Patel, V. P. & Lodish, H. F. J Cell Biol 102, 449-456 (1986); Sawada, K. et al., J Clin Invest 83, 1701-1709 (1989); Socolovsky, M. et al., Blood 92, 1491-1496 (1998);Terszowski, G. et al. Blood (2004); von Lindern, M. et al. Blood 94, 550-559 (1999); Waskow, C., et al., Blood 104, 1688-1695 (2004)). When quantifying erythropoietic growth in FIG. 14a, acridine orange histology and differential cell counts, rather than flow cytometry, were used to determine the PCE fraction in the harvested population. This method was chosen over flow cytometry to quantify erythropoietic growth and estimate the throughput of this in vitro assay because it provides a more robust estimate of the PCE fraction of a population.

| BACKGROUND CULTURE CONDITIONS | | | | | | | | | MEASURED EFFECT ON GROWTH | | | | | | |
|---|---|---|---|---|---|---|---|---|---|---|---|---|---|---|---|
| (“X” indicates the variable parameter found to affect growth) | | | | | | | | | | | High-Growth Condition | | Low-Growth Condition | | |
| | | harvest | | | [Fn] | [Dex] | [Ins] | [IGF-I] | Cell Type | | parameter | Avg | parameter | Avg | Sig |
| [Epo] (U/mL) | [SCF] (ng/mL) | time (h) | CO2 (%) | O2 (%) | (μg/sq cm) | (μM) | (μg/mL) | (ng/mL) | Ter-119 | CD71 | value | Growth +/− SD | value | Growth +/− SD | P< |
| X | 0 | 72 | 5 | 20 | 2 | 0 | 10 | 0 | + | − | [Epo] = 5 U/mL | 0.307 +/− 0.022 | [Epo] = 2 U/mL | 0.141 +/− 0.019 | 0.01 |
| X | 0 | 72 | 5 | 20 | 2 | 0 | 10 | 0 | + | − | [Epo] = 10 U/mL | 0.380 +/− 0.050 | [Epo] = 2 U/mL | 0.141 +/− 0.019 | 0.01 |
| X | 0 | 72 | 5 | 20 | 2 | 0 | 10 | 0 | + | − | [Epo] = 50 U/mL | 0.312 +/− 0.030 | [Epo] = 2 U/mL | 0.141 +/− 0.019 | 0.01 |
| X | 0 | 72 | 5 | 20 | 2 | 0 | 10 | 0 | + | − | [Epo] = 500 U/mL | 0.314 +/− 0.019 | [Epo] = 2 U/mL | 0.141 +/− 0.019 | 0.01 |
| 3.5 | X | 72 | 5 | 20 | 2 | 0 | 10 | 0 | + | + | [SCF] = 10 ng/mL | 1.512 +/− 0.100 | [SCF] = 0 ng/mL | 1.021 +/− 0.213 | 0.05 |
| 3.5 | X | 72 | 5 | 20 | 2 | 0 | 10 | 0 | + | − | [SCF] = 10 ng/mL | 0.202 +/− 0.023 | [SCF] = 0 ng/mL | 0.153 +/− 0.020 | 0.05 |
| 5 | X | 72 | 5 | 20 | 2 | 0 | 10 | 0 | + | + | [SCF] = 10 ng/mL | 1.448 +/− 0.155 | [SCF] = 0 ng/mL | 1.067 +/− 0.080 | 0.05 |
| 5 | X | 72 | 5 | 20 | 2 | 0 | 10 | 0 | + | + | [SCF] = 100 ng/mL | 1.929 +/− 0.154 | [SCF] = 0 ng/mL | 1.067 +/− 0.080 | 0.01 |
| 5 | X | 72 | 5 | 20 | 2 | 0 | 10 | 0 | + | + | [SCF] = 100 ng/mL | 1.929 +/− 0.154 | [SCF] = 1 ng/mL | 0.897 +/− 0.053 | 0.01 |
| 5 | X | 72 | 5 | 20 | 2 | 0 | 10 | 0 | + | − | [SCF] = 100 ng/mL | 0.369 +/− 0.041 | [SCF] = 1 ng/mL | 0.275 +/− 0.026 | 0.05 |
| 5 | X | 72 | 5 | 20 | 2 | 0 | 10 | 0 | + | + | [SCF] = 10 ng/mL | 1.448 +/− 0.155 | [SCF] = 1 ng/mL | 0.897 +/− 0.053 | 0.05 |
| 5 | X | 72 | 5 | 20 | 2 | 0 | 10 | 0 | + | + | [SCF] = 100 ng/mL | 1.929 +/− 0.154 | [SCF] = 10 ng/mL | 1.448 +/− 0.155 | 0.05 |
| 5 | X | 96 | 5 | 20 | 2 | 0 | 10 | 0 | + | − | [SCF] = 10 ng/mL | 0.608 +/− 0.081 | [SCF] = 1 ng/mL | 0.372 +/− 0.052 | 0.05 |
| 2 | 0 | X | 5 | 20 | 2 | 0 | 10 | 0 | + | + | harvest t = 48 h | 1.141 +/− 0.146 | harvest t = 72 h | 0.898 +/− 0.101 | 0.05 |
| 5 | 0 | X | 5 | 20 | 2 | 0 | 10 | 0 | + | + | harvest t = 72 h | 1.067 +/− 0.080 | harvest t = 96 h | 0.867 +/− 0.068 | 0.05 |
| 5 | 10 | X | 5 | 20 | 2 | 0 | 10 | 0 | + | + | harvest t = 72 h | 1.448 +/− 0.155 | harvest t = 96 h | 1.025 +/− 0.073 | 0.05 |
| 2 | 0 | X | 5 | 20 | 2 | 0 | 10 | 0 | + | − | harvest t = 72 h | 1.141 +/− 0.019 | harvest t = 48 h | −0.001 +/− 0.000 | 0.01 |
| 5 | 0 | X | 5 | 20 | 2 | 0 | 10 | 0 | + | − | harvest t = 96 h | 0.459 +/− 0.030 | harvest t = 72 h | 0.307 +/− 0.022 | 0.01 |
| 5 | 10 | X | 5 | 20 | 2 | 0 | 10 | 0 | + | − | harvest t = 96 h | 0.608 +/− 0.081 | harvest t = 72 h | 0.314 +/− 0.021 | 0.05 |
| 5 | 0 | 72 | X | 20 | 2 | 0 | 10 | 0 | + | + | CO2 = 5% | 2.373 +/− 0.233 | CO2 = 2.5% | 1.579 +/− 0.238 | 0.05 |
| 5 | 0 | 72 | X | 20 | 0, 2, 8 | 0 | 10 | 0 | + | + | CO2 = 5% | 2.095 +/− 0.307 | CO2 = 2.5% | 1.576 +/− 0.233 | 0.01 |
| 5 | 0 | 72 | X | 20 | 0 | 0 | 100 | 0 | + | + | CO2 = 5% | 2.096 +/− 0.121 | CO2 = 2.5% | 1.540 +/− 0.246 | 0.05 |
| 5 | 0 | 72 | X | 20 | 0, 2, 8 | 0 | 100 | 0 | + | + | CO2 = 5% | 2.696 +/− 0.660 | CO2 = 2.5% | 1.806 +/− 0.324 | 0.01 |
| 5 | 0 | 72 | 5 | X | 2 | 0 | 10 | 0 | + | + | Ox = 7.5% | 1.354 +/− 0.187 | Ox = 20% | 1.067 +/− 0.080 | 0.05 |
| 5 | 0 | 72 | 5 | X | 2 | 0 | 10 | 0 | + | − | Ox = 7.5% | 0.479 +/− 0.075 | Ox = 20% | 0.307 +/− 0.022 | 0.05 |
| 5 | 0 | 72 | 5 | 20 | X | 0 | 10 | 0 | + | + | [Fn] = 2 μg/sq | 2.373 +/− 0.233 | [Fn] = 0 μg/sq | 1.743 +/− 0.107 | 0.05 |
| | | | | | | | | | | | cm | | cm | | |
| 5 | 0 | 72 | 5 | 20 | X | 0 | 10 | 0 | + | + | [Fn] = 2 μg/sq | 2.168 +/− 0.036 | [Fn] = 0 μg/sq | 1.743 +/− 0.107 | 0.05 |
| | | | | | | | | | | | cm | | cm | | |
| 5 | 0 | 72 | 0.5 | 20 | 2 | X | 10 | 0 | + | − | [Dex] = 0 μM | 0.189 +/− 0.062 | [Dex] = 10 μM | 0.028 +/− 0.016 | 0.05 |
| 5 | 0 | 72 | 0.5 | 20 | 2 | X | 10 | 0 | + | − | [Dex] = 0 μM | 0.189 +/− 0.062 | [Dex] = 1 μM | 0.016 +/− 0.006 | 0.05 |
| 5 | 0 | 72 | 0.5 | 20 | 2 | X | 10 | 10 | + | + | [Dex] = 0 μM | 0.605 +/− 0.151 | [Dex] = 1 μM | 0.278 +/− 0.102 | 0.05 |
| 5 | 0 | 72 | 5 | 20 | 0 | 0 | X | 0 | + | + | [Ins] = 100 μg/mL | 2.096 +/− 0.121 | [Ins] = 10 μg/mL | 1.743 +/− 0.107 | 0.05 |
| 5 | 0 | 72 | 5 | 20 | 0 | 0 | X | 0 | + | − | [Ins] = 100 μg/mL | 0.427 +/− 0.015 | [Ins] = 10 μg/mL | 0.382 +/− 0.008 | 0.05 |

Table II. in vitro erythroid growth is modulated by physiologic erythropoietic stimuli. The culture conditions listed on the left-hand side of the table specify the environment in which a growth measurement was made. All soluble growth factors used in these experiments were removed from culture after 1 day. An X in this first section of the table indicates the variable parameter found to have a significant erythropoietic growth-effect. The measured effect of that parameter on erythropoietic growth is then given on the right-hand side of the table. In this right-hand section of the table, the first item listed is the defining surface phenotype of the erythropoietic population. The surface phenotype is followed by the high-growth condition and the resulting average growth (per Lin⁻ cell seeded) that was observed at that condition. Next are the comparison (low-growth) condition and the average growth that was observed at that condition. Finally, the level of significance (P value) is given. Note that the first 4 entries to this table were taken from FIG. 13; other entries were obtained from similar figures. P values were determined by the two-tailed t test, except for the 3 italicized P values, which were determined using the one-tailed t test.

These histology-based studies revealed that cultured populations fell into four general classes (subsets Ε, β, γ, δ in FIG. 14b) based on their exposure to Epo, Stem Cell Factor (SCF), and Dexamethasone (Dex). Populations exposed to both Epo and SCF (subset β) underwent the most growth, but included a high fraction of nucleated cells, whereas populations exposed to Epo and Dex without SCF (subset δ) underwent less growth, but resulted in populations containing a high PCE fraction that may be more amenable to high-throughput image analysis and scoring. Based on the highest level of growth observed, the number of assays that can be conducted in this system was estimated. The hind-legs of C57BL/6J mice yield 6.35×10⁵+/−1.84×10⁵ Lin⁻ cells (average +/− standard deviation, data not shown), and, at the maximum observed level of growth, 5.83+/−0.75 PCEs were grown per Lin⁻ cell (FIG. 14a). Given that 2000 PCEs are used to conduct each assay, it is calculated that approximately 1850 in vitro erythroid MN assays can be conducted using the hind-leg BM of a single mouse.

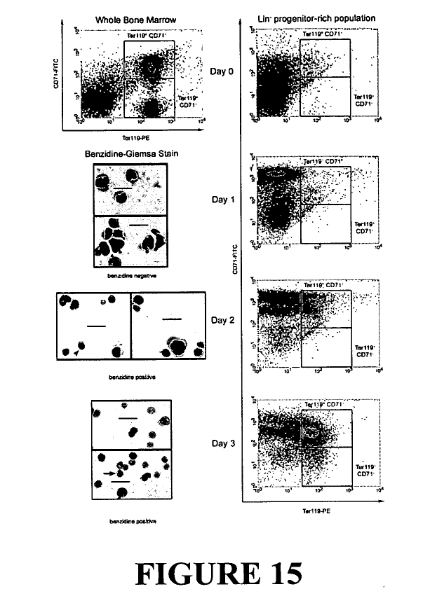

To confirm that erythropoiesis continued normally under these improved erythropoietic culture conditions, the cultured Lin⁻ BM was analyzed by flow cytometry and benzidine-Giemsa stain. It was found that large fractions of the population acquire Ter-119⁺, express hemoglobin, and enucleate (FIG. 15). However, culture under improved erythropoietic conditions yields a minor population that survives the entire culture period and expresses CD71, but never expresses Ter-119 or completes erythropoiesis. This population does not arise when Lin⁻ BM is cultured in the presence of Epo alone (FIG. 15 with FIG. 4), and it seems likely that this population arises from erythroid progenitors that temporarily proliferate in the presence of Epo and SCF before arresting growth when Epo and SCF are removed at day 1.

Finally, multi-linear regression was used to predict erythropoietic growth from the experimental design matrix (R²=0.95) and thus quantify the relative size of growth effects (see FIG. 16). P values for relating to parameter estimates in FIG. 16 are included in the Methods section below, under the heading “Estimation of scaled parameter effect estimates: method of multi-linear regression.” It was found that late-erythroid progenitors show a significant response to several physiologic stimuli in this culture system and that the largest eyrthropoietic growth effect was the primary Epo effect. The next largest effects were the secondary effect of Epo and SCF and the secondary effect of Epo and pO₂; these results indicate that growth in this system is largely controlled by well-established in vivo erythropoietic factors. This analysis differs from the above described analysis used to generate the data in FIG. 12. The main difference between the two manners is how the parameters were scaled, and the fact that this manner (FIG. 16) incorporates the data generated by culturing at 12% CO2.

### Example 3

### Effect of MGMT Expression on the BCNU Response

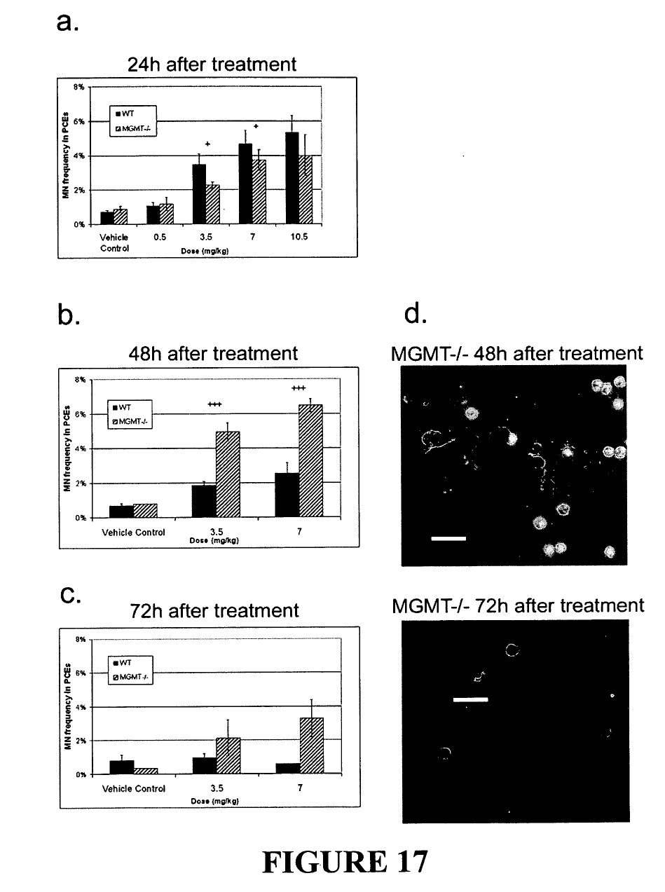

BCNU is an S_{N}1 alkylating agent that can form adducts at several nucleophilic sites on DNA, including the O⁶ position of Guanine (Singer, B. et al. Nature 276, 85-88 (1978); Bodell, W. J. Chem Res Toxicol 12, 965-970(1999); Ludlum, D. B. Mutat Res 233, 117-126 (1990)). After this initial addition reaction, the alkyl group on the modified DNA base can react a second time to form an interstrand crosslink, but the alkyl group can also be removed by an alkyl transferase known as O⁶-methylguanine DNA methyltransferase (MGMT) to repair the DNA (Kohn, K. W Cancer Res 37, 1450-1454 (1977); Samson, L. & Cairns, J Nature 267, 281-283 (1977)). When MGMT^{-/-} mice (on C57BL/6J background) were treated with intermediate doses of BCNU by intraperitoneal injection and were then examined by the in vivo MN test 24 h later, it was found that the MN frequency in PCEs was significantly lower than that observed in wild-type C57BL/6J mice (FIG. 17a). However, this trend had reversed 48 h after BCNU exposure, and the MN frequency was drastically higher in MGMT^{-/-} PCEs at these same intermediate exposure levels (FIG. 17b). In addition, this increased sensitivity in MGMT^{-/-} marrow continued to be evident 72 h after BCNU exposure; the MN frequency remained higher in MGMT^{-/-} PCEs at these same intermediate exposure levels (FIG. 17c), and the PCE:NCE ratio had decreased dramatically when BM samples from 48 h after exposure were compared to BM samples from 72 h after exposure (FIG. 17d), indicating that a high fraction of erythroid progenitors 3 d away from enucleation and reticulocyte formation had been killed. In BM samples from MGMT^{+/+} mice, the PCE:NCE ratio remained constant at various times following BCNU exposure. Taken together, these data indicate that erythroid progenitors in MGMT^{-/-} BM are more sensitive to BCNU, as measured by MN-formation and cell killing, than wild-type BM, and that PCE formation from damaged erythroid progenitors is delayed in MGMT^{-/-} BM.

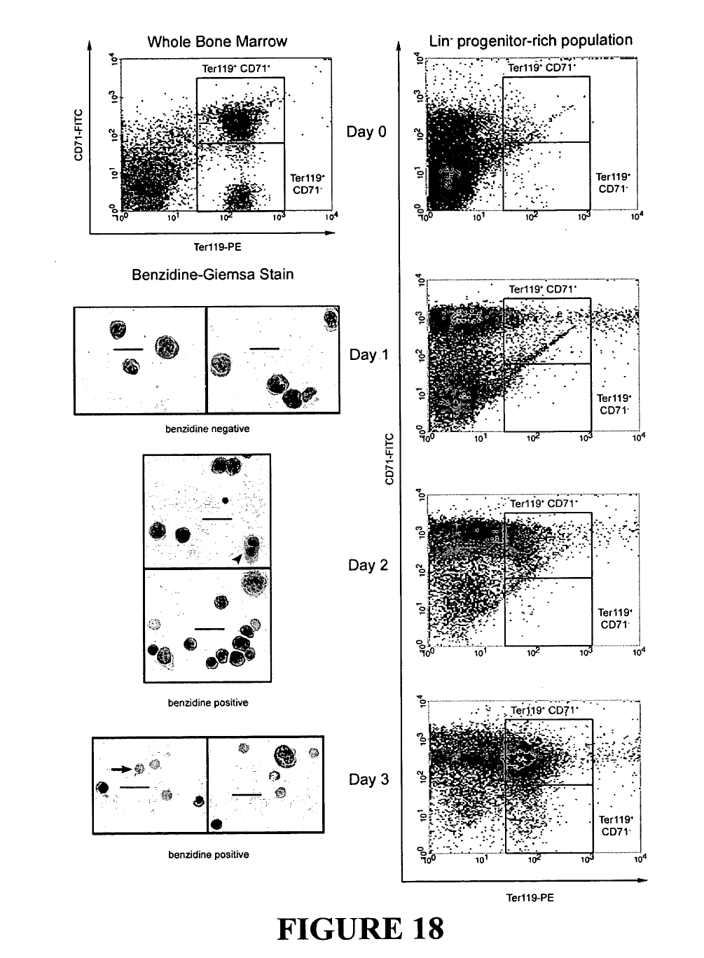

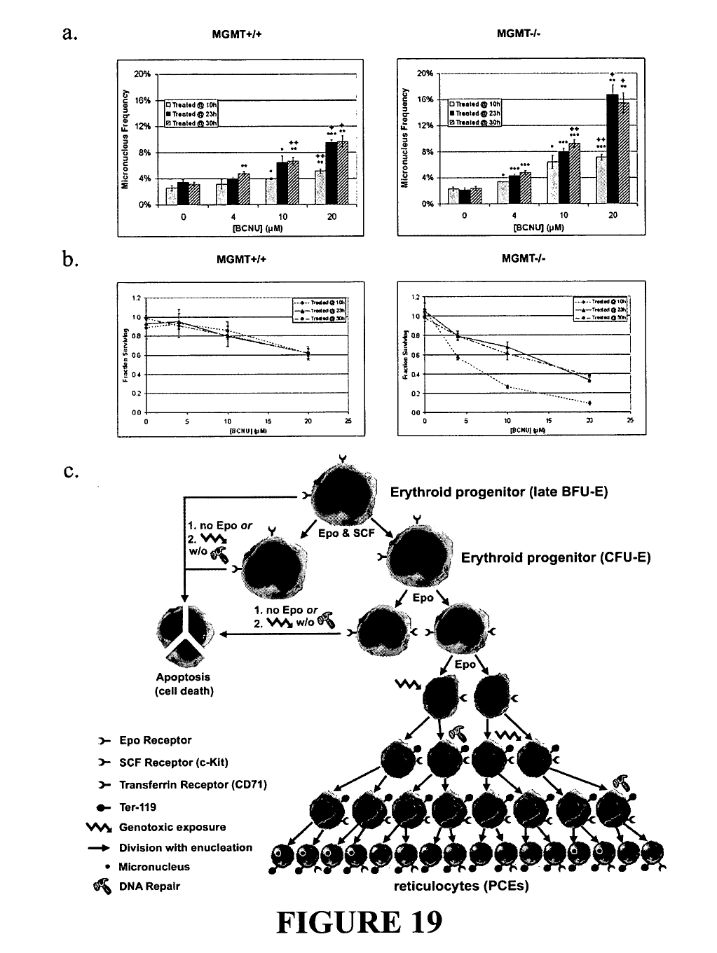

MGMT^{-/-} Lin⁻ BM was incorporated into erythropoietic culture to test whether the in vitro genotoxicity screen described here is capable of reflecting the DNA repair capacity of primary BM. In the absence of genotoxic exposure, the eythropoietic differentiation profile of the MGMT^{-/-} cultures (examined by flow cytometry and benzidine-Giemsa stain) was indistinguishable from that of wild-type cultures (compare FIG. 18 to FIG. 15). When BCNU was then introduced into these MGMT^{-/-} cultures, erythropoiesis continued normally before leading to significant increases in MN frequency as compared with wild-type cultures (FIG. 19a, see the 10 μM and 20 μM doses and the general trend). Furthermore, decreased cell survival was observed in the MGMT^{-/-} cultures, meaning that repair-deficient, late-erythroid progenitors are detectably more sensitive to BCNU both in terms of growth inhibition and in terms of microncucleated progeny production (FIG. 19a, b).

In FIG. 19a and FIG. 19b, BCNU was introduced into erythropoietic culture at various times after seeding (10 h, 23 h, and 30 h). In MGMT^{-/-} BM cultures, it was found that genotoxic treatment at earlier times led primarily to decreases in cell survival, whereas genotoxic treatment at later times led primarily to increases in the production of micronculeated PCEs. Furthermore, when MGMT^{+/+} BM cultures were treated 4 h after culture initiation, cell-killing dominated and very little MN formation was observed (data not shown). These observations indicate that a model like that shown in FIG. 19c might describe the effects of gentoxic exposure at various stages of late-erythropoiesis. Genetic damage to primitive progenitors, i.e. those that are sensitive to Epo stimulation on day 1, leads largely to apoptosis, while damage to the erythroid cells present in culture after Epo is removed leads largely to the formation of micronucleated PCEs.

Discussion

Although an erythropoietic culture system existed for mouse fetal liver, the use of human fetal tissue for genotoxicity testing is ethically complex and of questionable relevance. This work teaches a short-term erythropoietic culture system that used adult tissue for genotoxicity testing can be used. It was found that a fraction of Lin⁻ mouse BM undergoes normal erythropoiesis in short-term liquid culture while adhering to the established erythropoietic differentiation profile. However, the Lin⁻ BM population is heterogeneous and some of its members do not undergo erythropoiesis (FIG. 4, see CD71⁻/Ter-119⁻ cells in d1 flow cytometry data). These cells are not detectable in culture on d2 and d3 (FIG. 4), and cultured populations maintain >90% viability among objects with diameter >6 μm on d2 and d3 as determined by trypan blue exclusion. Therefore, it is likely that these non-erythropoietic cells undergo apoptosis and form debris sometime between d1 and d2.

By definition, Colony Forming Units-Erythroid (CFU-Es) can be stimulated with Epo to produce mature reticulocytes over 2-3 days. Therefore, the studies conducted in the culture system described here primarily measure the effect of growth factors, genotoxicants, and DNA-repair capacity on CFU-Es. This in vitro culture system provides a controlled and simplified version of in vivo erythropoiesis. It can be used in research involving CFU-Es. For example, the data of FIG. 14 show that hypoxic culture yields more PCE growth than normoxic culture provided that Epo is also present; these data suggest that CFU-Es might sense pO₂ directly in addition to sensing the kidneys' signal of hypoxia (Epo).

The differentiation profile of Lin⁻ BM cultured in improved conditions including SCF, Dex, and Insulin-like Growth Factor I along with Epo in the growth medium showed subtle changes from Epo-only cultures, possibly due to stimulated proliferation of late Burst Forming Units-Erythroid (BFU-Es) by SCF and Epo. These cells might then arrest near the CFU-E stage of differentiation when SCF and Epo are withdrawn 24 h after seeding (FIG. 19c). For example, perhaps by treating cultures shortly after seeding while increasing the duration of exposure to Epo beyond 24 h, further terminal division could be triggered to study the effect of dosing cells near the BFU-E stage of erythropoiesis.

This in vitro culture technique provides a novel experimental tool to study MN formation during erythropoiesis in a controlled manner. In this system all cells are Epo-stimulated during the first day of culture, and presumably erythropoietic cells metasynchronously undergo development. Normally, PCEs are sampled in the BM, rather than in the peripheral blood, partly to eliminate the complex influence that spleen function might have on the assay. The in vitro assay developed here eliminates splenic removal of microncucleated PCEs, allowing micronucleated PCEs to accumulate. This accumulation of genetically damaged PCEs might provide higher sensitivity and allow detection of mild genotoxic exposure. Furthermore, the in vitro erytroid MN assay described here is capable of detecting the DNA-repair capacity of the primary tissue donor. The BM of MGMT^{-/-} mice has been shown to display increased sensitivity to alkylating agents, and the in vivo MN data in this report are consistent with this observation (FIG. 17 ) (Glassner, B. J. et al. Mutagenesis 14, 339-347 (1999)). The in vitro erythropoietic system described here reflects the increased sensitivity observed in vivo (FIG. 19a and FIG. 19b). In this report, it is shown that an in vitro mouse assay can reflect the in vivo phenotype of the BM donor, suggesting that an in vitro human erythroid MN assay might better predict clinical hematopoietic genotoxicity.

Methods

Cells

Bone marrow cells were isolated from the hind legs of C57BL/6J mice aged 6-8 weeks (Jackson Laboratory, Bar Harbor, Me.) and were mechanically dissociated by pipetting in Iscove modified Dulbecco medium (IMDM) containing 4% FBS. Single-cell suspensions were prepared by passing the dissociated cells through 70 μm cell strainers. Bone marrow cells with diameter larger than 6 μm were counted using a Coulter particle counter Z1 (Beckman Coulter, Fullerton, Calif.).

Erythropoietic Culture

Total bone marrow cells were labeled with biotin-conjugated α-lineage-marker (α-Lin) antibodies, consisting of α-CD3e, α-CD11b, α-CD45R/B220, α-Ly6G/Ly6C, and α-TER-119 antibodies (2 μl each Ab: 10⁶ cells) (BD Pharmingen, San Diego, Calif.), and Lin⁻ cells were purified through a StemSep column as per the manufacturer's instructions (StemCell Technologies, Vancouver, BC, Canada). Purified cells were seeded in either fibronectin-coated (2 μg/cm²) or uncoated tissue-culture treated polystyrene wells (BD Discovery Labware, Bedford, Mass.), as indicated in the text, at a cell density of 10⁵/mL. On the first day, the purified cells were cultured in IMDM containing 15% FBS, 1% detoxified bovine serum albumin (BSA), 200 μg/mL holo-transferrin (Sigma, St Louis, Mo.), 10 μg/mL recombinant human insulin (Sigma), 2 mM L-glutamine, 10⁻⁴ M β-mercaptoethanol, and various soluble growth factors as indicated in the text. These additional growth factors included Epo (Amgen, Thousand Oaks, Calif.), SCF (R&D systems, Minneapolis, Minn.), IGF-I (R&D systems), and Dexamethasone (Sigma); these factors were added in various combinations (see text) in order to quantify their relative erythropoietic growth effects. On the second day, these media were replaced with erythroid-differentiation medium (EDM) (IMDM containing 20% FBS, 2 mM L-glutamine, and 10⁻⁴ M β-mercaptoethanol). At harvest, suspended cells were removed from culture wells by pipetting and then the culture well was incubating in phosphate-buffered saline (PBS)/10% FBS/5 mM EDTA (ethylenediaminetetraacetic acid) at 37° C, for 5 minutes to dissociate adherent cells. Dissociated cells were then removed by pipetting and combined with the suspended cell fraction from the same culture well.

Immunostaining and Flow Cytometry to Analyze Erythroid Differentiation

Bone marrow-derived cells were immunostained at 4° C. in PBS/4% FBS. Cells were incubated with phycoerythrin (PE)-conjugated α-Ter119 (1:200) (BD Pharmingen) and fluorescein isothiocyanante (FITC)-conjugated α-CD71 (1:200) (BD Pharmingen) antibodies for 15 minutes and were then washed in PBS/4% FBS. Flow cytometry was carried out on a Becton Dickinson FACSCalibur (Franklin Lakes, N.J.). Flow cytometry plots and region statistics were acquired using CellQuest Pro™ (BD Biosciences, San Jose, Calif.).

Cytospin Preparation and Histological Staining

Approximately 2×10⁴ cells per culture were centrifuged onto slides for 3 minutes at 800 rpm (Cytospin 3; Thermo Shandon, Pittsburgh, Pa.) and air dried. For benzidine-Giemsa staining, cells were fixed in −20° C. methanol for 2 minutes and stained with 3,3′-diaminobenzidine and Giemsa stains according to the manufacturer's recommendations (Sigma). For acridine orange staining, cells were fixed in room-temperature methanol for 10 minutes and stained in buffered acridine orange (20 μg/mL) (acridine orange [Fisher Scientific, Hanover Park, Ill.] in staining buffer [19 mM NaH₂PO₄ and 81 mM Na₂HPO₄]) for 10 minutes at 4° C. After acridine orange staining cells were protected from light, washed for 10 minutes in 4° C. staining buffer, air dried, and stored at 4° C. until microscopic examination and scoring was complete.

in vivo Dose Delivery and Histology

BCNU was first dissolved in cold absolute ethanol, and then mice were dosed with BCNU by intraperitoneal injection using a vehicle of 10% ethanol in PBS. At the appropriate time following dosage (24 h, 48 h, or 72 h), the mice were euthanized using CO₂, and the bone marrow was removed from the femurs and tibiae of the hind legs. A single cell suspension was generated by mechanical dissociation and this suspension was then passed through a cellulose column to remove most non-erythroid cells. This enriched erythroid population was then spread on a slide, fixed in methanol, and stained with Acridine Orange for histological examination. PCEs and MN-PCEs were enumerated by differential cell counting, and a minimum of 2000 PCEs was examined for each treated animal to determine the MN frequency in the bone marrow.

Dose Delivery to Erythropoietic Cultures

Lin⁻ bone marrow was cultured, according to the method described for in vitro erythroid differentiation, in 500 μL of medium per culture well. On the first day, the culture medium included Epo (10 U/mL), SCF (100 ng/mL), IGF-I (100 ng/mL), and Dexamethasone (10 μM) in addition to the basal supplements previously listed. Alkylating agents were added to the culture medium 23 hours after seeding, and were removed, along with soluble growth factors, by exchanging the medium for EDM one hour after dosing (24 hours after seeding). For dosing with BCNU (Sigma), BCNU was first dissolved in 4° C. ethanol (EtOH) to make a 10 mM solution. This 10 mM solution was then diluted in 4° C. IMDM to produce a 0.4 mM BCNU solution in IMDM/4% EtOH. A volume of this 0.4 mM BCNU solution (0-25 μL) was then added to each culture, along with a compensatory volume of vehicle (IMDM/4% EtOH), such that each culture was exposed to the targeted concentration of BCNU (0-20 μM) and an equal concentration of EtOH (0.2% by volume). For dosing with MNNG (Aldrich, Milwaukee, Wis.), MNNG was first dissolved in 4° C. IMDM to yield two solutions of different concentration (2 μg/mL and 10 μg/mL). To deliver the 0.02 μg/mL dose to cultures, 5 μL of 2 μg/mL MNNG was added to the medium; to deliver the 0.10 μg/mL or 0.20 μg/mL dose to cultures, 5 μL or 10 μL of 10 μg/mL MNNG was added to the medium, respectively. For dosing with MMS (Aldrich), MMS was first dissolved in 4° C. IMDM to yield two solutions of different concentration (0.5 mM and 5 mM). To deliver the 10 μM dose to cultures, 10 μL of 0.5 mM MMS was added to the medium; to deliver the 50 μM or 100 μM dose to cultures, 5 μL or 10 μL of 5 mM MMS were added to the medium, respectively. All solutions containing alkylating agents were prepared immediately before dosing to minimize degradation and decreases in reactivity.

Viable Cell Counting, Histological Imaging and Quantitation

Trypan blue-exclusion viable cell counts were conducted using a ViCellXR viable cell counter (Beckman Coulter, Miami, Fla.) according to the manufacturer's instructions. The instrument was set to include cells with a diameter between 6 μM and 50 μM in analyses. Slides were examined blind using a Labophot microscope (Nikon, Garden City, N.Y.) and representative micrographs were acquired using a Sony DSC-P93A Cyber-Shot digital camera. Micrographs of benzidine-Giemsa stained cells were acquired using a 100× oil-immersion objective and brightfield illumination, while micrographs of acridine orange stained cells were acquired using a 40× oil-immersion objective and fluorescence (100 W Hg lamp excitation). Histological slides were examined blind and differential cell counting was used to enumerate relevant cell types and thus quantify the % PCE among total cells and % MN-PCE among PCEs (>2000 cells scored per slide).

Estimation of Scaled Parameter Effect Estimates: Method of Multi-Linear Regression.

The following is a description of the method used to generate the model and parameter estimates reported in FIG. 16.

X̅≡the design matrix before scaling, with size n₁=144×n₂=8. n₁ is the number of experimental measurements that were made, while n₂ is the number of independent parameters (covariates) that were tested. For example, the first condition listed in FIG. 14a is represented thus:
X̅_{1,j}=[0,0,0,0,0,72,0.05,0.1995]

Each element in the original design matrix is then normalized such that each parameter ranges from 0 to 1, as shown below:

$${X}_{ij} = \frac{{χ}_{ij} - {min}_{i ∈ [ 1 , {n}_{1} ]} {χ}_{ij}}{{max}_{i ∈ [ 1 , {n}_{1} ]} {χ}_{ij} - {min}_{i ∈ [ 1 , {n}_{1} ]} {χ}_{ij}}$$

In this scaled design matrix, the first condition presented in FIG. 14a appears as:
X̅_{1,j}=[0,0,0,0,0,0,0,1]

Secondary interactions that increase the column rank of the design matrix are then incorporated as additional columns. For example, the elements representing the secondary interaction between Epo (j=1) and SCF (j=2) are added to the scaled design matrix as shown below:
X_{i,n}_{₂}₊₁=X_{i,1}*X_{i,2}

Ultimately, a scaled design matrix of size n₁=144×n₃=27 is obtained, where n₃ is the sum of the number of primary interaction (n₂) and the number of linearly-independent secondary interactions (for this experimental design, n₃-n₂=19). The vector of parameter estimates ( b̅) is then calculated, using multi-linear regression, to provide the best possible prediction of the experimentally measured growth-response vector ( y̅) using the design matrix ( X̅):
b̅=( X̅′ X̅)⁻¹ X̅′ y̅

These parameter estimates are then used to predict the growth response (\hat{y̅}):
X̅ b̅=\hat{y̅}

The root mean squared error (RMSE) and R² for the model are then calculated. Below, y_{avg} is the mean of the measured growth-response vector ( y̅).

$$RMSE = \frac{{\sum }_{i = 1}^{144} {[ {y}_{i} - {\hat{y}}_{i} ]}^{2}}{{n}_{1} - {n}_{3}}$$

$${R}^{2} = 1 - \frac{{\sum }_{i = 1}^{144} {[ {y}_{i} - {\hat{y}}_{i} ]}^{2}}{{\sum }_{i = 1}^{144} {[ {y}_{i} - {y}_{avg} ]}^{2}}$$

The error (εⱼ) and the t-ratio (εⱼ) for each parameter estimate are then calculated as:

$${ɛ}_{j} = RMSE * \sqrt{{( {\overline{\overline{X}}}^{′} \overline{\overline{X}} )}_{j , j}^{- 1}}$$

$${t}_{j} = \frac{{b}_{j}}{{ɛ}_{j}}$$

Finally, the upper and lower bound of the 95 percent confidence intervals for all parameters are calculated using the relevant t value (α=0.05, degrees of freedom=n1-n 3) thus:
upperⱼ=bⱼ+t(n₁−n₃)_{α/2}*εⱼ
lowerⱼ=bⱼ−t(n₁−n₃)_{α/2}*εⱼ

A summary of the results obtained from this complete model (n₃=27) is provided below:

| | | Stand- | 95% Confidence | | | |
|---|---|---|---|---|---|---|
| Model | Scaled | ard | Interval | | | |
| Parameter | Estimate | Error | Min | Max | t Ratio | P |
| Epo | 3.136 | 0.178 | 2.783 | 3.489 | 17.576 | <0.0001 |
| Epo * SCF | 1.648 | 0.147 | 1.358 | 1.938 | 11.242 | <0.0001 |
| Epo * O2 | −1.079 | 0.178 | −1.431 | −0.727 | −6.067 | <0.0001 |
| Epo * CO2 | −0.845 | 0.173 | −1.187 | −0.520 | −4.883 | <0.0001 |
| SCF * Dex | 0.451 | 0.147 | 0.160 | 0.741 | 3.073 | 0.003 |
| time * CO2 | 0.392 | 0.173 | 0.049 | 0.734 | 2.263 | 0.026 |
| SCF * O2 | −0.366 | 0.178 | −0.719 | −0.014 | −2.059 | 0.042 |
| SCF * CO2 | −0.351 | 0.173 | −0.694 | −0.009 | −2.031 | 0.045 |
| IGF * O2 | 0.349 | 0.178 | −0.003 | 0.701 | 1.962 | 0.052 |
| Dex | −0.298 | 0.151 | −0.597 | 0.002 | −1.967 | 0.052 |
| Dex * O2 | 0.272 | 0.178 | −0.080 | 0.625 | 1.531 | 0.129 |
| Epo * Fn | 0.244 | 0.147 | −0.046 | 0.535 | 1.667 | 0.098 |
| IGF-I | −0.231 | 0.151 | −0.530 | 0.069 | −1.526 | 0.129 |
| SCF * IGF-I | 0.186 | 0.147 | −0.105 | 0.476 | 1.267 | 0.208 |
| CO2 | −0.166 | 0.228 | −0.618 | 0.286 | −0.727 | 0.469 |
| SCF | 0.166 | 0.162 | −0.155 | 0.487 | 1.023 | 0.308 |
| CO2 * IGF-I | 0.096 | 0.173 | −0.247 | 0.438 | 0.552 | 0.582 |
| time | −0.090 | 0.138 | −0.364 | 0.183 | −0.654 | 0.514 |
| O2 * Fn | 0.081 | 0.178 | −0.272 | 0.433 | 0.453 | 0.651 |
| O2 * time | −0.077 | 0.178 | −0.429 | 0.275 | −0.434 | 0.665 |
| Epo * IGF-I | 0.072 | 0.147 | −0.218 | 0.362 | 0.491 | 0.624 |
| Fn | 0.053 | 0.138 | −0.221 | 0.326 | 0.380 | 0.705 |
| EPO * Dex | −0.049 | 0.147 | −0.339 | 0.242 | −0.331 | 0.741 |
| CO2 * Fn | 0.038 | 0.173 | −0.304 | 0.381 | −0.221 | 0.826 |
| O2 | 0.010 | 0.191 | −0.368 | 0.389 | 0.055 | 0.956 |
| CO2 * Dex | −0.004 | 0.173 | −0.347 | 0.338 | −0.025 | 0.980 |
| Epo * time | 0.002 | 0.147 | −0.287 | 0.292 | 0.012 | 0.990 |

R² = 0.9508
R²_{adj} = 0.9398
RMSE = 0.4478

Some of the less significant factors (those highlighted above) were then removed from the design matrix in order to simplify the model and emphasize the more important parameter effects. Specifically, all effects with |tⱼ|<1 were removed from the final version of the model (represented in Fig H) except for the time and Fn effects. These primary effects were retained, despite their low t-ratios, because they were key effects in the original experimental design. In this manner, the adjusted R² value was slightly improved and the model was simplified. A summary of the results obtained from this reduced model (n₃=17) is provided below:

| | | Stand- | 95% Confidence | | | |
|---|---|---|---|---|---|---|
| Model | Scaled | ard | Interval | | | |
| Parameter | Estimate | Error | Min | Max | t Ratio | P |
| Epo | 3.130 | 0.141 | 2.852 | 3.409 | 22.259 | <0.0001 |
| Epo * SCF | 1.668 | 0.138 | 1.396 | 1.940 | 12.122 | <0.0001 |
| Epo * O2 | −1.075 | 0.156 | −1.384 | −0.766 | −6.883 | <0.0001 |
| Epo * CO2 | −0.888 | 0.144 | −1.173 | −0.604 | −6.183 | <0.0001 |
| SCF * Dex | 0.471 | 0.138 | 0.198 | 0.743 | 3.420 | 0.007 |
| SCF * CO2 | −0.395 | 0.144 | −0.679 | −0.111 | −2.749 | 0.007 |
| IGF * O2 | 0.376 | 0.145 | 0.089 | 0.663 | 2.589 | 0.011 |
| SCF * O2 | −0.362 | 0.156 | −0.671 | −0.053 | −2.317 | 0.022 |
| Dex | −0.333 | 0.130 | −0.590 | −0.077 | −2.571 | 0.011 |
| time * CO2 | 0.320 | 0.134 | 0.056 | 0.585 | 2.395 | 0.018 |
| Epo * Fn | 0.264 | 0.138 | −0.008 | 0.537 | 1.921 | 0.057 |
| Dex * O2 | 0.256 | 0.145 | −0.031 | 0.544 | 1.767 | 0.080 |
| SCF * IGF-I | 0.206 | 0.138 | −0.067 | 0.478 | 1.495 | 0.137 |
| IGF-I | −0.200 | 0.130 | −0.456 | 0.057 | −1.540 | 0.126 |
| SCF | 0.138 | 0.147 | −0.154 | 0.429 | 0.934 | 0.352 |
| time | −0.124 | 0.082 | −0.286 | 0.038 | −1.515 | 0.132 |
| Fn | 0.096 | 0.092 | −0.087 | 0.278 | 1.037 | 0.302 |

R² = 0.9501
R²_{adj} = 0.9438
RMSE = 0.4330

Statistics, Design of Experiments, and Multi-Linear Regression

All independent sample t-tests and P value calculations for mean comparisons were conducted using the data analysis tool in Microsoft® Excel (assuming unequal variances). The design of experiments feature in JMP 5 (SAS Institute, Inc., Cary, N.C.) was used to generate the resolution IV, minimum-aberration, 2-level, 6-factor (2_{IV}⁶⁻²) fractional-factorial design used to estimate primary and secondary parameter effects. This 2_{IV}⁶⁻² fractional-factorial design was conducted in 3 different atmospheres using a single well-mixed Lin⁻ BM population to estimate the primary and secondary effects of 8 factors (6 original parameters, pO₂, and pCO₂) simultaneously and thus minimize the inherent variability of primary cell isolations. A model to predict the measured erythropoietic growth was generated using multi-linear regression (see directly above), and MATLAB® 6.5 (The MathWorks) a technical computing language and interactive environment, was used to perform all matrix algebra, statistical calculations, and to generate FIG. 16a.

### REFERENCES

1. Ferguson, L. R. and A. E. Pearson, The clinical use of mutagenic anticancer drugs. Mutat Res, 1996. 355(1-2): p. 1-12.
2. Aebi, S., et al., Loss of DNA mismatch repair in acquired resistance to cisplatin. Cancer Res, 1996. 56(13): p. 3087-90.
3. Branch, P., et al., Spontaneous development of drug resistance: mismatch repair and p53 defects in resistance to cisplatin in human tumor cells. Oncogene, 2000. 19(28): p. 3138-45.
4. Carethers, J. M., et al., Mismatch repair proficiency and in vitro response to 5-fluorouracil. Gastroenterology, 1999. 117(1): p. 123-31.
5. Casorelli, I., et al., Drug treatment in the development of mismatch repair defective acute leukemia and myelodysplastic syndrome. DNA Repair (Amst), 2003. 2(5): p. 547-59.
6. Fedier, A., et al., Resistance to topoisomerase poisons due to loss of DNA mismatch repair. Int J Cancer, 2001. 93(4): p. 571-6.
7. Reese, J. S., L. Liu, and S. L. Gerson, Repopulating defect of mismatch repair-deficient hematopoietic stem cells. Blood, 2003. 102(5): p. 1626-33.
8. Swann, P. F., et al., Role of postreplicative DNA mismatch repair in the cytotoxic action of thioguanine. Science, 1996. 273(5278): p. 1109-11.
9. Tokunaga, E., et al., Differential growth inhibition by 5-fluorouracil in human colorectal carcinoma cell lines. Eur J Cancer, 2000. 36(15): p. 1998-2006.
10. Fiumicino, S., et al., Sensitivity to DNA cross-linking chemotherapeutic agents in mismatch repair-defective cells in vitro and in xenografts. Int J Cancer, 2000. 85(4): p. 590-6.
11. Ames, B. N., W. E. Durston, et al. (1973). “Carcinogens are mutagens: a simple test system combining liver homogenates for activation and bacteria for detection.” Proc Natl Acad Sci USA 70(8): 2281-5.
12. Ames, B. N., J. McCann, et al. (1975). “Methods for detecting carcinogens and mutagens with the Salmonella/mammalian-microsome mutagenicity test.” Mutat Res 31(6): 347-64. Ashby, J. and R. Mohammed (1986). “Slide preparation and sampling as a major source of variability in the mouse micronucleus assay.” Mutat Res 164(4): 217-35.
13. Bechensteen, A. G., S. Halvorsen, et al. (1994). “Recombinant human insulin-like growth factor 1 (rh-IGF-1) stimulates erythropoiesis in adult, but not in newborn mice.” Acta Physiol Scand 151(1): 117-23.
14. Casarett, L. J., J. Doull, et al. (2001). Casarett and Doull's toxicology: the basic science of poisons. New York, McGraw-Hill Medical Pub. Division.
15. Dertinger, S. D., D. K. Torous, et al. (1996). “Simple and reliable enumeration of micronucleated reticulocytes with a single-laser flow cytometer.” Mutat Res 371(3-4): 283-92.
16. Dertinger, S. D., D. K. Torous, et al. (1997). “Flow cytometric analysis of micronucleated reticulocytes in mouse bone marrow.” Mutat Res 390(3): 257-62.
17. DiMasi, J. A., R. W. Hansen, et al. (2003). “The price of innovation: new estimates of drug development costs.” J Health Econ 22(2): 151-85.
18. Fenech, M. (2000). “The in vitro micronucleus technique.” Mutat Res 455(1-2): 81-95.
19 Fenech, M. and A. A. Morley (1985). “Measurement of micronuclei in lymphocytes.” Mutat Res 147(1-2): 29-36.
20. Fibach, E., D. Manor, et al. (1989). “Proliferation and maturation of human erythroid progenitors in liquid culture.” Blood 73(1): 100-3.
21. Fibach, E. and E. A. Rachmilewitz (1993). “The two-step liquid culture: a novel procedure for studying maturation of human normal and pathological erythroid precursors.” Stem Cells 11 Suppl 1: 36-41.
22. Galloway, S. M. (2004). Personal Communication.
23. Giarratana, M. C., L. Kobari, et al. (2005). “Ex vivo generation of fully mature human red blood cells from hematopoietic stem cells.” Nat Biotechnol 23(1): 69-74.
24. Glassner, B. J., G. Weeda, et al. (1999). “DNA repair methyltransferase (Mgmt) knockout mice are sensitive to the lethal effects of chemotherapeutic alkylating agents.” Mutagenesis 14(3): 339-47.
25. Hayashi, M., T. Sofuni, et al. (1983). “An application of Acridine Orange fluorescent staining to the micronucleus test.” Mutat Res 120(4): 241-7.
26. Heddle, J. A., M. C. Cimino, et al. (1991). “Micronuclei as an index of cytogenetic damage: past, present, and future.” Environ Mol Mutagen 18(4): 277-91.
27. Ishikawa, Y. and T. Ito (1988). “Kinetics of hemopoietic stem cells in a hypoxic culture.” Eur J Haematol 40(2): 126-9.
28. Kina, T., K. Ikuta, et al. (2000). “The monoclonal antibody TER-119 recognizes a molecule associated with glycophorin A and specifically marks the late stages of murine erythroid lineage.” Br J Haematol 109(2): 280-7.
29. Kirkland, D. J., D. G. Gatehouse, et al. (1990). Basic mutagenicity tests: UKEMS recommended procedures: UKEMS Sub-Committee on Guidelines for Mutagenicity Testing: report, part 1. Cambridge [England]; New York, Cambridge University Press.
30. Kolber, M. A., K. O. Broschat, et al. (1990). “Cytochalasin B induces cellular DNA fragmentation.” Faseb J 4(12): 3021-7.
31. Koller, M. R., J. G. Bender, et al. (1992). “Reduced oxygen tension increases hematopoiesis in long-term culture of human stem and progenitor cells from cord blood and bone marrow.” Exp Hematol 20(2): 264-70.
32. LaIuppa, J. A., E. T. Papoutsakis, et al. (1998). “Oxygen tension alters the effects of cytokines on the megakaryocyte, erythrocyte, and granulocyte lineages.” Exp Hematol 26(9): 835-43.
33. Lodish, H. F. (2003). Molecular cell biology. New York, W.H. Freeman and Company.
34. Loken, M. R., V. O. Shah, et al. (1987). “Flow cytometric analysis of human bone marrow: I. Normal erythroid development.” Blood 69(1): 255-63.
35. Malgor, L. A., L. Barrios, et al. (1987). “Erythropoietic action of dexamethasone on the anemia associated with an experimental chronic renal failure.” Acta Physiol Pharmacol Latinoam 37(3): 365-76.
36. McAdams, T. A., W. M. Miller, et al. (1998). “pH is a potent modulator of erythroid differentiation.” Br J Haematol 103(2): 317-25.
37. Meulemans, A., B. Giroux, et al. (1989). “Permeability of two nitrosoureas, carmustine and fotemustine in rat cortex.” Chemotherapy 35(5): 313-9.
38. Morita, T., N. Asano, et al. (1997). “Evaluation of the rodent micronucleus assay in the screening of IARC carcinogens (groups 1, 2A and 2B) the summary report of the 6th collaborative study by CSGMT/JEMS MMS. Collaborative Study of the Micronucleus Group Test. Mammalian Mutagenicity Study Group.” Mutat Res 389(1): 3-122.
39. Okumura, N., K. Tsuji, et al. (1992). “Changes in cell surface antigen expressions during proliferation and differentiation of human erythroid progenitors.” Blood 80(3): 642-50.
40. Patel, V. P. and H. F. Lodish (1986). “The fibronectin receptor on mammalian erythroid precursor cells: characterization and developmental regulation.” J Cell Biol 102(2): 449-56.
41. Peschel, C., G. Konwalinka, et al. (1987). “Changes of the phenotype of erythroid progenitor cells (BFU-E, CFU-E) and their progenies during early steps of differentiation.” Br J Haematol 65(2): 131-5.
42. Podesta, M., G. Piaggio, et al. (2001). “Modified in vitro conditions for cord blood-derived long-term culture-initiating cells.” Exp Hematol 29(3): 309-14.
43. Romagna, F. and C. D. Staniforth (1989). “The automated bone marrow micronucleus test.” Mutat Res 213(1): 91-104.
44. Sakatoku, H. and S. Inoue (1997). “In vitro proliferation and differentiation of erythroid progenitors of cord blood.” Stem Cells 15(4): 268-74.
45. Sancar, A., L. A. Lindsey-Boltz, et al. (2004). “Molecular mechanisms of mammalian DNA repair and the DNA damage checkpoints.” Annu Rev Biochem 73: 39-85.
46. Sawada, K., S. B. Krantz, et al. (1989). “Human colony-forming units-erythroid do not require accessory cells, but do require direct interaction with insulin-like growth factor I and/or insulin for erythroid development.” J Clin Invest 83(5): 1701-9.
47. Sawada, K., S. B. Krantz, et al. (1987). “Purification of human erythroid colony-forming units and demonstration of specific binding of erythropoietin.” J Clin Invest 80(2): 357-66.
48. Schmid, W. (1973). “Chemical mutagen testing on in vivo somatic mammalian cells.” Agents Actions 3(2): 77-85.
49. Schmid, W. (1975). “The micronucleus test.” Mutat Res 31(1): 9-15.
50. Socolovsky, M., A. E. Fallon, et al. (1998). “The prolactin receptor rescues EpoR−/− erythroid progenitors and replaces EpoR in a synergistic interaction with c-kit.” Blood 92(5): 1491-6.
51. Socolovsky, M., H. Nam, et al. (2001). “Ineffective erythropoiesis in Stat5a(−/−)5b(−/−) mice due to decreased survival of early erythroblasts.” Blood 98(12): 3261-73.
52. Styles, J. A., H. Clark, et al. (2001). “Automation of mouse micronucleus genotoxicity assay by laser scanning cytometry.” Cytometry 44(2): 153-5.
53. Terszowski, G., C. Waskow, et al. (2004). “Prospective isolation and global gene expression analysis of the colony-forming unit-erythrocyte (CFU-E).” Blood.
54. Tinwell, H. and J. Ashby (1989). “Comparison of acridine orange and Giemsa stains in several mouse bone marrow micronucleus assays—including a triple dose study.” Mutagenesis 4(6): 476-81.
55. von Lindern, M., W. Zauner, et al. (1999). “The glucocorticoid receptor cooperates with the erythropoietin receptor and c-Kit to enhance and sustain proliferation of erythroid progenitors in vitro.” Blood 94(2): 550-9.
56. Waskow, C., G. Terszowski, et al. (2004). “Rescue of lethal c-KitW/W mice by erythropoietin.” Blood 104(6): 1688-95.
57. Zhang, J. and H. F. Lodish (2004). “Constitutive activation of the MEK/ERK pathway mediates all effects of oncogenic H-ras expression in primary erythroid progenitors.” Blood 104(6): 1679-87.
58. Zhang, J., M. Socolovsky, et al. (2003). “Role of Rα s signaling in erythroid differentiation of mouse fetal liver cells: functional analysis by a flow cytometry-based novel culture system.” Blood 102(12): 3938-46.

All references described herein are incorporated by reference.

We claim:

1. A method for determining the genotoxic effect of a test compound on an erythroid cell comprising:
a) culturing in vitro a starting population of cells, wherein said population of cells contains erythroid progenitors, for a sufficient time and under sufficient conditions to obtain erythropoietic growth;
b) adding at least one test compound to the culture medium of step (a); and
c) harvesting the differentiated erythroid populations, and measuring at least one of the following characteristics: (i) total number and presence of micronuclei (MN) in the PCEs, wherein the presence of greater level of MN in the cells relative to a control population of cells not exposed to the test compound indicates the genotoxic effect of said test compound; (ii) total cell number of erythroid-specific cells, wherein a decrease in total cell number of erythroid-specific cell numbers provides and indication of a general cytotoxic effect of said test compound; and (iii) PCE number, size or shape, wherein a change in PCE number, size, or shape provides an indication of the efficacy of said test compound in treating a given erythropoietic defect.

2. The method of claim 1, wherein the erythroid progenitor is selected from the group consisting of cells near the colony-forming unit erythroid (CFU-E) stage of erythropoiesis, the burst-forming unit erythroid (BFU-E) stage of erythropoiesis, the CFU-granulocyte erythroid macrophage megakarocyte (CFU-GEMM) stage of erythropoiesis, and long-term repopulating hematopoietic stem cell (LT-HSC) and combinations thereof.

3. The method of claim 1, wherein the starting population of cells is substantially free of mature granulocytes, reticulocytes, macrophages, T cells, B cells, and erythrocytes.

4. The method of claim 1, wherein the starting population of cells is substantially free of differentiated erythrocytes.

5. The method of claim 1, wherein the starting population of cells is isolated from a human.

6. The method of claim 1, wherein the starting population of cells is isolated by selecting erythroid progenitors from a population of human cells which express at least two surface markers selected from the group consisting of CD34, CD41, CD71 and CD36, or wherein the starting population of cells is a lineage marker negative (Lin−) population of human cells.

7. The method of claim 5, wherein the starting population of cells is the mononuclear cell (MNC) fraction of human peripheral blood.

8. The method of claim 7, wherein the starting population of cells does not express at least one of the cell surface markers selected from the group consisting of Gr-1, Mac-1, CD3, B-220, Ter-119, Lin, Sca-1, IL7-Rα, and CD41.

9. The method of claim 7, wherein the starting population of cells expresses c-Kit and CD71.

10. The method of claim 1, wherein the starting population of cells is cultured in an initial culture medium which enhances proliferation of the starting population of cells.

11. The method of claim 10 wherein the initial culture medium includes an additional factor selected from the group consisting of erythropoietin, holotransferrin, dexamethasone, stem cell factor, insulin, and IGF-1.

12. The method of claim 1, wherein the test compound is added to the culture medium for 12-24 hours, after which the cells are washed to remove the test compound and fresh culture medium is added.

13. The method of claim 12, wherein the fresh culture medium promotes the erythroid differentiation of the erythroid progenitor cells into terminally differentiated erythrocytes.

14. The method of claim 1, wherein the test compound is any compound to which a human can be exposed.

15. The method of claim 14, wherein the test compound is selected from the group consisting of pharmaceuticals, diagnostics, pesticides, cosmetics, vaccines, lotions, foods, agro-chemicals, nanoparticles, commodity chemicals, chemical intermediates, biomaterials and packing materials.

16. The method of claim 15, wherein the test compound is a candidate compound for use in treating an erythropoietic developmental defect, and the number of MN-PCEs and the number, size, and shape of PCEs are compared between control cultures and cultures exhibiting an erythropoietic developmental disorder.

17. The method of claim 16, wherein the erythropoietic developmental defect is selected from the group consisting of sickle cell anemia, thalassemias, polycythemia vera, and other myeloproliferative disorders.

18. The method of claim 1, wherein the genotoxic effect is a clastogenetic effect or an aneugenetic effect.

19. The method of claim 1, wherein the percentage of cells comprising micronuclei is determined by a method selected from the group consisting of flow cytometry, histological analysis and scoring, automated image analysis platforms, and biochemical analyses.

20. A method for screening a group of test compounds to determine the genotoxic effect of each individual test compound on erythroid cells, comprising:
a) selecting at least four individual test compounds to comprise the group of test compounds; and
b) determining the genotoxic effect of each individual test compound on an erythroid cell using the method of claim 1.

21. The method of claim 20, wherein the group of test compounds is screened simultaneously in a series of parallel cultures.

22. The method of claim 20, wherein each genotoxic effect of an individual test compound is determined at multiple concentrations for that compound.

23. The method of claim 20, wherein the group of test compounds comprises at least 30 different individual test compounds.

24. The method of claim 20, wherein each genotoxic effect of an individual test compound is determined at multiple concentrations for that compound.
